SEQUENCE LISTING (37 sequences)

SEQ ID NO: 1 - protein, 583 aa, Homo sapiens
MKLLHVFLLFLCFHLRFCKVTYTSQEDLVEKKCLAKKYTHLSCDKVFCQPWQRCIEGTCVCKLPYQCPKNGTAVCATNRRSFPTYCQQKSLECLHPGTKFLNNGTCTAEGKFSVSLKHGNTDSEGIVEVKLVDQDKTMFICKSSWSMREANVACLDLGFQQGADTQRRFKLSDLSINSTECLHVHCRGLETSLAECTFTKRRTMGYQDFADVVCYTQKADSPMDDFFQCVNGKYISQMKACDGINDCGDQSDELCCKACQGKGFHCKSGVCIPSQYQCNGEVDCITGEDEVGCAGFASVTQEETEILTADMDAERRRIKSLLPKLSCGVKNRMHIRRKRIVGGKRAQLGDLPWQVAIKDASGITCGGIYIGGCWILTAAHCLRASKTHRYQIWTTVVDWIHPDLKRIVIEYVDRIIFHENYNAGTYQNDIALIEMKKDGNKKDCELPRSIPACVPWSPYLFQPNDTCIVSGWGREKDNERVFSLQWGEVKLISNCSKFYGNRFYEKEMECAGTYDGSIDACKGDSGGPLVCMDANNVTYVWGVVSWGENCGKPEFPGVYTKVANYFDWISYHVGRPFISQYNV

SEQ ID NO: 2 - protein, 20 aa, Mus musculus
METDTLLLWVLLLWVPGSTG

SEQ ID NO: 3 - protein, 18 aa, Homo sapiens
MKLLHVFLLFLCFHLRFC

SEQ ID NO: 4 - skipped

SEQ ID NO: 5 - protein, 565 aa, Homo sapiens
KVTYTSQEDLVEKKCLAKKYTHLSCDKVFCQPWQRCIEGTCVCKLPYQCPKNGTAVCATNRRSFPTYCQQKSLECLHPGTKFLNNGTCTAEGKFSVSLKHGNTDSEGIVEVKLVDQDKTMFICKSSWSMREANVACLDLGFQQGADTQRRFKLSDLSINSTECLHVHCRGLETSLAECTFTKRRTMGYQDFADVVCYTQKADSPMDDFFQCVNGKYISQMKACDGINDCGDQSDELCCKACQGKGFHCKSGVCIPSQYQCNGEVDCITGEDEVGCAGFASVTQEETEILTADMDAERRRIKSLLPKLSCGVKNRMHIRRKRIVGGKRAQLGDLPWQVAIKDASGITCGGIYIGGCWILTAAHCLRASKTHRYQIWTTVVDWIHPDLKRIVIEYVDRIIFHENYNAGTYQNDIALIEMKKDGNKKDCELPRSIPACVPWSPYLFQPNDTCIVSGWGREKDNERVFSLQWGEVKLISNCSKFYGNRFYEKEMECAGTYDGSIDACKGDSGGPLVCMDANNVTYVWGVVSWGENCGKPEFPGVYTKVANYFDWISYHVGRPFISQYNV

SEQ ID NO: 6 - protein, 6 aa, synthetic construct
GGSSGG

SEQ ID NO: 7 - protein, 585 aa, Homo sapiens [sequence omitted]

SEQ ID NO: 8-11 - skipped (4 entries)

SEQ ID NO: 12 - protein, 260 aa, synthetic construct
KLSCGVKNRMHIRRKRIVGGKRAQLGDLPWQVAIKDASGITCGGIYIGGCWILTAAHCLRASKTHRYQIWTTVVDWIHPDLKRIVIEYVDRIIFHENYNAGTYQNDIALIEMKKDGNKKDCELPRSIPACVPWSPYLFQPNDTCIVSGWGREKDNERVFSLQWGEVKLISNCSKFYGNRFYEKEMECAGTYDGSIDACKGDSGGPLVCMDANNVTYVWGVVSWGENCGKPEFPGVYTKVANYFDWISYHVGRPFISQYNV

SEQ ID NO: 13-15 - skipped (3 entries)

SEQ ID NO: 16 - protein, 1135 aa, synthetic construct
KVTYTSQEDLVEKKCLAKKYTHLSCDKVFCQPWQRCIEGTCVCKLPYQCPKNGTAVCATNRRSFPTYCQQKSLECLHPGTKFLNNGTCTAEGKFSVSLKHGNTDSEGIVEVKLVDQDKTMFICKSSWSMREANVACLDLGFQQGADTQRRFKLSDLSINSTECLHVHCRGLETSLAECTFTKRRTMGYQDFADVVCYTQKADSPMDDFFQCVNGKYISQMKACDGINDCGDQSDELCCKACQGKGFHCKSGVCIPSQYQCNGEVDCITGEDEVGCAGFASVTQEETEILTADMDAERRRIKSLLPKLSCGVKNRMHIRRKRIVGGKRAQLGDLPWQVAIKDASGITCGGIYIGGCWILTAAHCLRASKTHRYQIWTTVVDWIHPDLKRIVIEYVDRIIFHENYNAGTYQNDIALIEMKKDGNKKDCELPRSIPACVPWSPYLFQPNDTCIVSGWGREKDNERVFSLQWGEVKLISNCSKFYGNRFYEKEMECAGTYDGSIDACKGDSGGPLVCMDANNVTYVWGVVSWGENCGKPEFPGVYTKVANYFDWISYHVGRPFISQYNVGSSGGKVTYTSQEDLVEKKCLAKKYTHLSCDKVFCQPWQRCIEGTCVCKLPYQCPKNGTAVCATNRRSFPTYCQQKSLECLHPGTKFLNNGTCTAEGKFSVSLKHGNTDSEGIVEVKLVDQDKTMFICKSSWSMREANVACLDLGFQQGADTQRRFKLSDLSINSTECLHVHCRGLETSLAECTFTKRRTMGYQDFADVVCYTQKADSPMDDFFQCVNGKYISQMKACDGINDCGDQSDELCCKACQGKGFHCKSGVCIPSQYQCNGEVDCITGEDEVGCAGFASVTQEETEILTADMDAERRRIKSLLPKLSCGVKNRMHIRRKRIVGGKRAQLGDLPWQVAIKDASGITCGGIYIGGCWILTAAHCLRASKTHRYQIWTTVVDWIHPDLKRIVIEYVDRIIFHENYNAGTYQNDIALIEMKKDGNKKDCELPRSIPACVPWSPYLFQPNDTCIVSGWGREKDNERVFSLQWGEVKLISNCSKFYGNRFYEKEMECAGTYDGSIDACKGDSGGPLVCMDANNVTYVWGVVSWGENCGKPEFPGVYTKVANYFDWISYHVGRPFISQYNV

SEQ ID NO: 17-20 - skipped (4 entries)

SEQ ID NO: 21 - protein, 1156 aa, synthetic construct
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGSSGGKVTYTSQEDLVEKKCLAKKYTHLSCDKVFCQPWQRCIEGTCVCKLPYQCPKNGTAVCATNRRSFPTYCQQKSLECLHPGTKFLNNGTCTAEGKFSVSLKHGNTDSEGIVEVKLVDQDKTMFICKSSWSMREANVACLDLGFQQGADTQRRFKLSDLSINSTECLHVHCRGLETSLAECTFTKRRTMGYQDFADVVCYTQKADSPMDDFFQCVNGKYISQMKACDGINDCGDQSDELCCKACQGKGFHCKSGVCIPSQYQCNGEVDCITGEDEVGCAGFASVTQEETEILTADMDAERRRIKSLLPKLSCGVKNRMHIRRKRIVGGKRAQLGDLPWQVAIKDASGITCGGIYIGGCWILTAAHCLRASKTHRYQIWTTVVDWIHPDLKRIVIEYVDRIIFHENYNAGTYQNDIALIEMKKDGNKKDCELPRSIPACVPWSPYLFQPNDTCIVSGWGREKDNERVFSLQWGEVKLISNCSKFYGNRFYEKEMECAGTYDGSIDACKGDSGGPLVCMDANNVTYVWGVVSWGENCGKPEFPGVYTKVANYFDWISYHVGRPFISQYNV

SEQ ID NO: 22 - skipped

SEQ ID NO: 23 - protein, 603 aa, Mus musculus
MKLAHLSLFLLALHLSSSRSPSASDLPQEELVDQKCLLQKYTHRSCNKVFCQPWQRCIEGTCICKLPYQCPRAGTPVCAMNGRSYPTYCHQKSFECLHPEIKFSHNGTCAAEGKFNVSLIYGRTKTEGLVQVKLVDQDERMFICKNSWSMAEANVACVDLGFPLGVRDIQGSFNISGNLHINDTECLHVHCRGVETSLAECAFTKRRTELSNGLAGVVCYKQDADFPTSLSFQCVNGKHIPQEKACNGVNDCGDQSDELCCKGCRGNASLCKSGVCIPDQYKCNGEVDCITGEDESRCEEDRQQNIPKGLARSAQGEAEIETEETEMLTPGMDNERKRIKSLLPKLSCGVKRNTHTRRKRVIGGKPANVGDYPWQVAIKDGQRITCGGIYIGGCWILTAAHCVRPSRAHSYQVWTALLDWLKPNSQLGIQTVKRVIVHEKYNGATFQNDIALIEMKMHTGKKECELPNSVPACVPWSPYLFQPNDRCIISGWGRGKDNQKVYSLRWGEVDLIGNCSQFYPDRYYEKEMQCAGTRDGSIDACKGDSGGPLVCEDINNVTYVWGIVSWGENCGKPEFPGVYTRVANYFDWISYHVGRSLVSQHNV

SEQ ID NO: 24 - protein, 7 aa, Homo sapiens
MDANNVT

SEQ ID NO: 25 - protein, 8 aa, Homo sapiens
PFISQYNV

SEQ ID NO: 26 - protein, 10 aa, Homo sapiens
REKDNERVFS

SEQ ID NO: 27 - protein, 12 aa, synthetic construct
NTASSGADYPDE

SEQ ID NO: 28 - protein, 9 aa, Homo sapiens
RPFISQYNV

SEQ ID NO: 29 - protein, 13 aa, synthetic construct
GGGGGGGGGGGGG

SEQ ID NO: 30 - protein, 4 aa, Homo sapiens
DGNK

SEQ ID NO: 31 - protein, 10 aa, synthetic construct
CAGFASVAQE

SEQ ID NO: 32 - protein, 26 aa, Mus musculus
CEEDRQQNIPKGLARSAQGEAEIETE

SEQ ID NO: 33 - protein, 6 aa, Homo sapiens
KVTYTS

SEQ ID NO: 34 - protein, 9 aa, Mus musculus
RSPSASDLP

SEQ ID NO: 35 - protein, 80 aa, synthetic construct
  REPEAT 1..4: note = single repeat of GGSS
  VARIANT 1..80: note = up to 19 copies of GGSS can be deleted
GGSSGGSSGGSSGGSSGGSSGGSSGGSSGGSSGGSSGGSSGGSSGGSSGGSSGGSSGGSSGGSSGGSSGGSSGGSSGGSS

SEQ ID NO: 36 - protein, 82 aa, synthetic construct
  REPEAT 1..4: note = single repeat of GGSS
  VARIANT 1..80: note = up to 19 copies of GGSS can be deleted
GGSSGGSSGGSSGGSSGGSSGGSSGGSSGGSSGGSSGGSSGGSSGGSSGGSSGGSSGGSSGGSSGGSSGGSSGGSSGGSSGG

SEQ ID NO: 37 - protein, 120 aa, synthetic construct
  REPEAT 1..6: note = single repeat of GGSSGG
  VARIANT 1..120: note = up to 19 copies of GGSSGG can be deleted
GGSSGGGGSSGGGGSSGGGGSSGGGGSSGGGGSSGGGGSSGGGGSSGGGGSSGGGGSSGGGGSSGGGGSSGGGGSSGGGGSSGGGGSSGGGGSSGGGGSSGGGGSSGGGGSSGGGGSSGG

(19) United States
(12) Patent Application Publication
(10) Pub. No.: US 2025/0009855 A1
(43) Pub. Date: Jan. 9, 2025

(54) COMPLEMENT FACTOR-I FORMULATIONS

(71) Applicant: Vertex Pharmaceuticals Incorporated, Boston, MA (US)

(72) Inventors: Bing SHAN, San Jose, CA (US); Brian David CONNOLLY, Berkeley, CA (US); Sibu BALAN, Redwood City, CA (US); Anju CHATTERJI, Martinez, CA (US)

(21) Appl. No.: 18/750,927

(22) Filed: Jun. 21, 2024

Related U.S. Application Data
(63) Continuation of application No. PCT/US2022/082179, filed on Dec. 21, 2022.
(60) Provisional application No. 63/293,013, filed on Dec. 22, 2021.

(51) Int. Cl. A61K 38/48

(52) U.S. Cl. CPC A61K 38/482; C12Y 304/21045

(57) ABSTRACT

Provided herein are pharmaceutically acceptable formulations comprising wild type Complement Factor I (CFI) and variants thereof; in some embodiments the wild type CFI and variants thereof are part of a fusion construct, e.g. a fusion construct comprising human serum albumin. The formulations stabilize CFI against acute stresses during storage in either a liquid or lyophilized state. Also provided are methods of making the formulations, and methods of using the formulations in the treatment of diseases.

### CROSS REFERENCE TO RELATED APPLICATION

This application claims priority to U.S. Provisional Application No. 63/293,013 filed on Dec. 22, 2021, the contents of which are incorporated herein by reference in their entireties.

### REFERENCE TO AN ELECTRONIC SEQUENCE LISTING

The contents of the electronic sequence listing (VTEX_707_01WO_SeqList_ST26.xml; Size: 31,757 bytes; and Date of Creation: Dec. 17, 2022) are herein incorporated by reference in its entirety.

### BACKGROUND

Complement Factor I (CFI) is a soluble protein of the complement system, that regulates complement activation by cleaving cell-bound or fluid phase C3b and C4b. It is a soluble glycoprotein that circulates in human blood and acts to maintain the balance between the classical, lectin, and alternative pathways of the complement system. Dysregulated CFI, mutated and dysfunctional CFI, and CFI deficiency have been implicated in diseases involving the complement system. Needed are pharmaceutically acceptable CFI formulations useful for regulating complement system. Provided here are formulations address this need.

### SUMMARY

The disclosure provides formulations comprising wild type complement factor I (CFI) or CFI variants, and fusion proteins thereof. The formulations provided stabilize the active ingredient against acute stresses and allow for storage in both liquid and lyophilized states. Also provided are methods of making the formulations, and methods of using the formulations in the treatment of diseases.

### DETAILED DESCRIPTION

The disclosure provides formulations comprising wild type CFI or CFI variants, and fusion proteins thereof. The formulations provided stabilize the active ingredient against acute stresses and allow for storage in both liquid and lyophilized states. Also provided are methods of making the formulations, and methods of using the formulations in the treatment of diseases.

### I. Pharmaceutically Acceptable Formulations

The pharmaceutically acceptable formulations described herein provide stabilizing properties to wild type CFI or CFI variants, and fusion constructs thereof of the disclosure (interchangeably referred to herein here as the “active ingredient” or “active pharmaceutical ingredient”) at a range of concentrations that allow for pharmaceutically acceptable storage conditions. Stabilizing properties can be, for example, prevention of degradation, maintenance of concentration, prevention of aggregation, and/or maintenance of bioactivity.

The formulations of the disclosure include wild type CFI, CFI variants, fusion constructs comprising wild type CFI (e.g. CFI-HSA), or fusion constructs comprising a CFI variants (e.g. any CFI variant of Table 2-HSA fusion), buffering agents, tonicity modifiers, surfactants, and further optionally bulking agents, cryoprotectants, lyoprotectants, and/or stabilizers.

The formulation can be designed to support storage of the active ingredient as a solid (dry) form (e.g. lyophilized cake or cryopreserved). In some embodiments, the formulation is a lyophilizate. In some embodiments, the formulation is a liquid formulation.

The formulations can be designed to support storage (while maintaining, e.g. stability, activity) of the active ingredient in solution as a liquid at a range of concentrations. The concentration can be adjusted in the formulation for use in different types of administration (e.g. subcutaneous or intravenous). For example, the active ingredient can be stored in the formulation as a liquid solution at about 10 mg/mL to about 300 mg/mL, for example at about 10 mg/ml, about 15 mg/mL, 20 mg/mL, about 25 mg/mL, 30 mg/mL, about 35 mg/mL, 40 mg/mL, about 45 mg/mL, about 50 mg/mL, about 55 mg/mL, about 60 mg/mL, about 65 mg/mL, about 70 mg/mL, about 75 mg/mL, about 80 mg/mL, about 85 mg/mL, about 90 mg/mL, about 95 mg/mL, about 100 mg/mL, about 105 mg/mL, about 110 mg/mL, about 115 mg/mL, about 120 mg/mL, about 125 mg/mL, about 130 mg/mL, about 135 mg/mL, about 140 mg/mL, about 145 mg/mL, about 150 mg/mL, about 155 mg/mL, about 160 mg/mL, about 165 mg/mL, about 175 mg/mL, about 180 mg/ml, about 185 mg/mL, about 190 mg/mL, about 195 mg/mL, about 200 mg/mL, about 205 mg/mL, about 210 mg/mL, about 215 mg/mL, about 220 mg/mL, about 225 mg/ml, about 230 mg/mL, about 235 mg/mL, about 245 mg/mL, at about 250 mg/mL, at about 255 mg/mL, at about 260 mg/mL, at about 260 mg/mL, at about 265 mg/mL, at about 270 mg/mL, at about 275 mg/mL, at about 280 mg/mL, at about 285 mg/mL, at about 290 mg/mL, at about 295 mg/mL, or about 300 mg/mL. In some embodiments, the formulation comprises the active ingredient at a concentration of about 50 mg/mL. In some embodiments, the formulation comprises the active ingredient at a concentration of about 100 mg/mL. In some embodiments, the formulation comprises the active ingredient at a concentration greater than 150 mg/mL. In some embodiments, the formulation comprises the active ingredient at a concentration of about 170 mg/ml. In some embodiments, the formulation comprises the active ingredient at a concentration of about 190 mg/mL.

The formulations of the disclosure allow the active ingredient to maintain stability (e.g. activity) at any one or more of the following temperatures: −80° C.+/−2° C., −20° C.+/−2° C., 0° C.+/−2° C., 4° C.+/−2° C., 25° C.+/−2° C., 37° C.+/−2° C., 45° C.+/−2° C., or at 60° C.+/−2° C.

The formulations of the disclosure allow the active ingredient to maintain stability (e.g. activity) for at least 1 week, at least 2 weeks, at least 3 weeks, at least 1 month, at least 2 months, at least 3 months, at least 4 months, at least 5 months, at least 6 months, at least 7 months, at least 8 months, at least 9 months, at least 10 months, at least 11 months, at least one year, at least 2 years, or more.

The pH of the formulation described herein is any pH that provides stabilizing properties to the CFI variants and fusion constructs described herein. In some embodiments, the formulation comprises a pH between about 5 to about 7.5. The pH can be, for example, about pH 5.0, about pH 5.1, about pH 5.2, about pH 5.3, about pH 5.4, about pH 5.5, about pH 5.6, about pH 5.7, about pH of 5.8, about pH 5.9, about pH 6.0, about pH 6.1, about pH 6.2, about pH 6.2, about pH 6.3, about pH 6.4, about pH 6.5, about pH 6.6, about pH 6.7, about pH 6.8, about pH 6.9, about pH 7, about pH 7.1, about pH 7.2, about pH 7.3, about pH 7.4, or about pH 7.5.

The formulations include a pharmaceutically acceptable buffering agent. Buffer agents can be used to maintain the pH of the formulation in a desired range. Any suitable buffer can be used in the formulations of the disclosure. Examples of pharmaceutically acceptable buffers comprise histidine, acetate, citrate, succinate, tartrate, glutamate, glycine, bicarbonate, sulfate, nitrate, phosphate, and hydroxymethylaminomethane (tris) buffers. In some embodiments the buffering agent is present at about 1 mM to about to about 50 mM. In some embodiments the buffering agent is present at about 20 nM. In some embodiments, the buffer comprises histidine, e.g. histidine hydrochloride. In some embodiments, the buffer comprises acetate, e.g. sodium acetate. In some embodiments, the buffer comprises succinate, e.g. sodium succinate.

The formulations include a surfactant, sometimes referred to as a wetting and/or solubilizing agent. Without limitation, a surfactant excipient can be used to modulate solubility and bioavailability of biologic molecules, increasing the stability of biologics molecules in dosage forms, and maintain preferred polymorphic forms. Examples of surfactants include polysorbate 20 (PS20, Tween 20), polysorbate 80 (PS80, Tween 80), PS80/20, poloxamer (Pluronic F68 and F127), Triton X-100, Brij 30, and Brij 35. In some embodiments, the formulation of the disclosure comprises a surfactant. In some embodiments, the surfactant is polysorbate 20 or polysorbate 80. In some embodiments, the formulation comprises polysorbate 20. In some embodiments, the polysorbate 20 is about 0.01% to about 0.05%. In some embodiments, the polysorbate is about 0.02%. In some embodiments, the formulation comprises polysorbate 80. In some embodiments, the polysorbate 80 is about 0.01% to about 0.05%. In some embodiments, the polysorbate 80 is about 0.02%.

The formulations include a tonicity modifier, sometimes may also be referred to as a tonicity agent, tonicifier or tonicifying agent. Tonicity modifiers are used to adjust the osmolality of the formulation such that the osmotic pressure of the formulation when administered to a subject does not cause any deleterious effects, such as unwanted cell lysis. Any pharmaceutically acceptable tonicity modifiers that is compatible with the active ingredients of the disclosure may be suitable for use in the formulations described herein. Pharmaceutically acceptable tonicity modifiers can include, for example, mannitol, sorbitol, lactose, dextrose, trehalose, sodium chloride, potassium chloride, glycerol, glycerin, or arginine hydrochloride. In some embodiments, the formulation comprises a tonicity modifiers. In some embodiments, the tonicity modifier is arginine hydrochloride. In some embodiments, the formulation comprises arginine hydrochloride. In some embodiments, the arginine hydrochloride is about 10 mM to about 200 mM. In some embodiments, the arginine hydrochloride is about 70 mM. In some embodiments, the arginine hydrochloride is about 135 mM. In some embodiments sorbitol is the tonicity modifier, and the sorbitol is present at about 1% to about 10% v/v, e.g. at about 5% v/v. In some embodiments the tonicity modifier is trehalose, and trehalose is present at about 1% to about 15% v/v, e.g. at about 10% v/v. In some embodiments the tonicity modifier is sodium chloride, and is present at about 1 mM to about 500 mM, e.g. at about 150 mM. In some embodiments the tonicity modifier is sorbitol or trehalose for non-ionic buffering agents, and sodium chloride for ionic buffering agents.

The formulation can include a bulking agent, for example a trehalose or glycine. In some embodiments, the bulking agent is glycine and is present in the formulation at about 1% to about 5% v/v, e.g. at about 2%. In some embodiments, the bulking agent is glycine and is present in the formulation at about 1 mM to about 100 nM, e.g. at about 60 mM.

The formulation can include a cryoprotectant and/or a lyoprotectant. Cryoprotectants and lyoprotectants are included in formulations to provide protection to biologic molecules from denaturation during cycles of freezing and thawing. They can also aide in preserving biologic integrity, e.g. maintaining bioactivity, during lyophilization (i.e. freeze drying) and reconstitution. In some instances, cryoprotectants and lyoprotectants can be referred to interchangeably. Any pharmaceutically acceptable cryoprotectant and/or lyoprotectant capable of providing stabilizing properties to formulations including the CFI variants and fusion constructs of the disclosure can be used. Non-limiting examples of cryoprotectants and lyoprotectants include polyols, disaccharides, polysaccharides, glucose, glycine, mannitol, sorbitol, sucrose, trehalose, and dextran 40. In some embodiments, the formulation comprises a cryoprotectant. In some embodiments, the cryoprotectant is at least one of sucrose, glycine, sorbitol, trehalose, and mannitol. In some embodiments, the cryoprotectant is sucrose. In some embodiments, the sucrose is about 2% to about 10%. In some embodiments, the sucrose is about 4% to about 5%. In some embodiments, the sucrose is about 8.5%. In some embodiments, the cryoprotectant is glycine. In some embodiments, the glycine is about 50 mM to about 150 mM, e.g. about 120 mM. In some embodiments, the glycine is about 120 mM. In some embodiments, the cryoprotectant is sorbitol. In some embodiments, the glucose is present in the formulation at about 1% to about 10% v/v, e.g. at about 4% v/v. In some embodiments, the sorbitol is present at about 1% to about 5%. In some embodiments, the sorbitol is about 2.5% v/v. In some embodiments, the trehalose is present at about 1% to about 10% v/v, e.g. about 4% v/v. In some embodiments, the sorbitol is about 2.5%. In some embodiments, the cryoprotectant is mannitol. In some embodiments, the mannitol is about 1 mM to about 100 mM. In some embodiments, the mannitol is about 60 mM.

The formulation can include a stabilizing agent, sometimes referred to as a stabilizer. A stabilizing agent is any excipient that provides stabilizing properties to formulations that include the CFI variants and fusion constructs described herein. For example, a stabilizing agent can be calcium chloride (CaCl₂)), histamine, methionine, ascorbic acid, glutathione, vitamin E, poly(ethylenimine), chelating agents, antimicrobial preservatives, antioxidant preservatives, or any other pharmaceutically acceptable stabilizing agent. In some embodiments the formulation comprises a stabilizer. In some embodiments, the stabilizer is calcium chloride. In some embodiments, the formulation comprises calcium chloride. In some embodiments, the calcium chloride is about 20 mM to about 50 mM. In some embodiments, the calcium chloride is about 35 mM.

Exemplary formulations are provided in Table 2.2 and Table 3.3.

In some embodiments the formulation comprises 20 mM sodium acetate, 5% sorbitol, 0.02% PS80, and is at a pH of about 5.

In some embodiments the formulation comprises 20 mM sodium acetate, 5% sorbitol, 0.02% PS80, and is at a pH of about 5.5.

In some embodiments the formulation comprises 20 mM sodium acetate, 150 mM sodium chloride, 0.02% PS80, and is at a pH of about 5.5.

In some embodiments the formulation comprises 20 mM sodium acetate, 70 mM arginine hydrochloride, 2% sucrose, 60 mM glycine, 0.02% PS80, and is at a pH of about 5.5.

In some embodiments the formulation comprises 20 mM histidine hydrochloride, 70 mM arginine hydrochloride, 2% sucrose, 60 mM glycine, 0.02% PS80, and is at a pH of about 6.

In some embodiments the formulation comprises 20 mM histidine hydrochloride, 150 mM sodium chloride, 0.02% PS80, and is at a pH of about 6.

In some embodiments the formulation comprises 20 mM sodium succinate, 10% trehalose, 0.02% PS80, and is at a pH of about 5.

In some embodiments the formulation comprises 20 mM sodium succinate, 5% trehalose, 2% glycine, 0.02% PS80, and is at a pH of about 5.

In some embodiments the formulation comprises 20 mM histidine hydrochloride, 10% trehalose, 0.02% PS80, and is at a pH of about 6.

In some embodiments the formulation comprises 20 mM histidine hydrochloride, 5% trehalose, 2% glycine, 0.02% PS80, and is at a pH of about 6.

In some embodiments the formulation comprises 20 mM histidine hydrochloride, 10% trehalose, 0.02% PS80, and is at a pH of about 7.

In some embodiments the formulation comprises 20 mM histidine hydrochloride, 5% trehalose, 2% glycine, 0.02% PS80, and is at a pH of about 7.

In some embodiments, the formulation comprises 20 mM histidine, 135 mM arginine hydrochloride, 0.02% polysorbate 20, and has a pH of about 5.8.

In some embodiments, the formulation comprises 20 mM histidine, 70 mM arginine hydrochloride, 4% sucrose, 0.02% polysorbate 20, and has pH of about 5.8.

In some embodiments, the formulation comprises 20 mM histidine, 8.5% sucrose, 0.02% polysorbate 20, and has pH of about 5.8.

In some embodiments, the formulation comprises 20 mM histidine, 70 mM arginine hydrochloride, 120 mM glycine, 0.02% polysorbate 20, and has a pH of about 5.8.

In some embodiments, the formulation comprises 20 mM histidine, 70 mM arginine hydrochloride, 2.5% sorbitol, 0.02% polysorbate 20, and has a pH of about 5.8.

In some embodiments, the formulation comprises 20 mM histidine, 70 mM arginine hydrochloride, 4% trehalose, 0.02% polysorbate 20, and has a pH of about 5.8.

In some embodiments, the formulation comprises 20 mM histidine, 5% sucrose, 35 mM calcium chloride, 0.02% polysorbate 20, and has a pH of about 5.8.

### II. Complement Factor I Variants and Fusion Constructs for Formulation

### A. Complement Factor I Variants

Provided herein are formulations that comprise variants comprising one or more modifications with respect to a wild type CFI, referred to herein as “CFI variants.” As used herein, a “modification” to a wild type CFI includes: a deletion of one or more amino acid residues, a deletion of one or more domains, a substitution of one or more amino acid residues, an insertion (i.e. addition) of one or more amino acid residues, an insertion (i.e. addition) of one or more domains, an inversion of or one or more domains, and a substitution of one or more domains.

The CFI variants of the disclosure do not directly act on C3, for example, the variants of the disclosure do not directly cleave C3, do not directly inhibit C3, do not directly inhibit the activation of C3, and do not directly reduce the activation of C3.

As used herein, a “wild type CFI” refers to any naturally occurring full-length CFI that is not a disease-causing CFI, with or without a signal sequence, and which may be of any species.

In some embodiments, a wild type CFI is plasma-derived. In some embodiments, a wild type CFI is a human wild type CFI. In some embodiments, a wild type, human CFI having a signal sequence comprises the amino acid sequence set forth in SEQ ID NO: 1 (as shown in Table 1 below). In some embodiments, a wild type CFI is a human CFI. In some embodiments, a wild type, human CFI does not include a signal sequence. In some embodiments, a wild type CFI without a signal sequence comprises the amino acid sequence set forth in SEQ ID NO: 5 (as shown in Table 1 below).

A wild type CFI comprises a heavy chain and a light chain, which are also referred to as the A-chain and B-chain, respectively. The heavy chain (A-chain) has four domains: the FI membrane attack complex (FIMAC) domain (residues 36 to 90 of SEQ ID NO: 5), the SRCR domain is further composed of a plurality of scavenger receptor cysteine-rich (SRCR) domains, a low density lipoprotein 1 domain (LDLr1), and a low density lipoprotein 2 domain (LDLr2). The light chain (B-chain) consists of a serine protease domain (SPD). The interface between these chains is referred to as the A:B chain interface.

A CFI variant of the disclosure includes one or more of a deletion of one or more amino acid residues of a wild type CFI, a deletion of one or more CFI domains of a wild type CFI, a substitution of one or more amino acid residues of a wild type CFI, an insertion of one or more amino acid residues to a wild type CFI, an inversion of one or more CFI domains of a wild type CFI, and an insertion of one or more domains to a wild type CFI.

The CFI variants of the disclosure may be generated by the introduction of one or more modifications to a CFI base molecule, wherein the domains of the CFI base molecule correspond to those domains found in a wild type CFI. A CFI base molecule may therefore be a wild type CFI of any species, or a CFI base molecule may comprise only portions of a wild type CFI, having only some of the domains of a wild type CFI of any species (e.g. already a CFI variant). In some embodiments, a CFI base molecule is a wild type, mouse CFI. In some embodiments, a CFI base molecule is a wild type, human CFI. In some embodiments, a CFI base molecule is a wild type, non-human primate CFI. In some embodiments, a CFI base molecule comprises only some domains of a wild type, human CFI.

In some embodiments, the CFI variants provided herein modulate the activity of the complement system and have at least one improved characteristic as compared to a wild type CFI. Such improved characteristics include, but are not limited to an increase or decrease in any one or more of bioavailability, half-life, activity, potency, catalytic capability, cofactor affinity (e.g. affinity for Factor H and/or CR1), substrate specificity and substrate affinity (e.g. affinity for C3b and/or C4b). In some embodiments, the improved characteristic is increased half-life. In some embodiments, the improved characteristic is an increase in activity, discussed further in detail, in subsequent sections below. In other embodiments, the improved characteristic is a change in substrate specificity for C3b and/or C4b, allowing for tunability of the CFI variant.

Provided in Table 1 are exemplary base molecules that may be used for the generation of CFI variants. The base molecules provided herein may be useful for modulation of the complement system without further modification, or may be useful for modulation of the complement system with further modification. For example, any one of the base molecules provided in Table 1 may be further modified to include one or more modifications, such as a deletion of one or more amino acid residues, a deletion of one or more CFI domains, a substitution of one or more amino acid residues, or an addition of one or more amino acid residues or CFI domains. The base molecules of Table 1 may be further part of a fusion construct, further described below.

TABLE 1
Base Molecules for Generation of CFI Variants

| | Nomenclature | |
|---|---|---|
| Description of CFI Base | of Base | |
| Molecule | Molecule | Amino Acid Sequence |
| Plasma derived, wild type | CFI-PD | MKLLHVFLLFLCFHLRFCKVTYTSQEDLVEKKCL |
| human CFI (wt hCFI) | | AKKYTHLSCDKVFCQPWQRCIEGTCVCKLPYQCP |
| (with signal sequence | | KNGTAVCATNRRSFPTYCQQKSLECLHPGTKFLN |
| underlined) | | NGTCTAEGKFSVSLKHGNTDSEGIVEVKLVDQDK |
| | | TMFICKSSWSMREANVACLDLGFQQGADTQRRF |
| | | KLSDLSINSTECLHVHCRGLETSLAECTFTKRRTM |
| | | GYQDFADVVCYTQKADSPMDDFFQCVNGKYISQ |
| | | MKACDGINDCGDQSDELCCKACQGKGFHCKSGV |
| | | CIPSQYQCNGEVDCITGEDEVGCAGFASVTQEETE |
| | | ILTADMDAERRRIKSLLPKLSCGVKNRMHIRRKRI |
| | | VGGKRAQLGDLPWQVAIKDASGITCGGIYIGGCW |
| | | ILTAAHCLRASKTHRYQIWTTVVDWIHPDLKRIVI |
| | | EYVDRIIFHENYNAGTYQNDIALIEMKKDGNKKD |
| | | CELPRSIPACVPWSPYLFQPNDTCIVSGWGREKDN |
| | | ERVFSLQWGEVKLISNCSKFYGNRFYEKEMECAG |
| | | TYDGSIDACKGDSGGPLVCMDANNVTYVWGVVS |
| | | WGENCGKPEFPGVYTKVANYFDWISYHVGRPFIS |
| | | QYNV (SEQ ID NO: 1) |
| wt hCFI, no signal | hCFI | KVTYTSQEDLVEKKCLAKKYTHLSCDKVFCQPW |
| sequence | | QRCIEGTCVCKLPYQCPKNGTAVCATNRRSFPTY |
| | | CQQKSLECLHPGTKFLNNGTCTAEGKFSVSLKHG |
| | | NTDSEGIVEVKLVDQDKTMFICKSSWSMREANVA |
| | | CLDLGFQQGADTQRRFKLSDLSINSTECLHVHCR |
| | | GLETSLAECTFTKRRTMGYQDFADVVCYTQKAD |
| | | SPMDDFFQCVNGKYISQMKACDGINDCGDQSDEL |
| | | CCKACQGKGFHCKSGVCIPSQYQCNGEVDCITGE |
| | | DEVGCAGFASVTQEETEILTADMDAERRRIKSLLP |
| | | KLSCGVKNRMHIRRKRIVGGKRAQLGDLPWQVA |
| | | IKDASGITCGGIYIGGCWILTAAHCLRASKTHRYQI |
| | | WTTVVDWIHPDLKRIVIEYVDRIIFHENYNAGTYQ |
| | | NDIALIEMKKDGNKKDCELPRSIPACVPWSPYLFQ |
| | | PNDTCIVSGWGREKDNERVFSLQWGEVKLISNCS |
| | | KFYGNRFYEKEMECAGTYDGSIDACKGDSGGPL |
| | | VCMDANNVTYVWGVVSWGENCGKPEFPGVYTK |
| | | VANYFDWISYHVGRPFISQYNV (SEQ ID NO: 5) |
| Δ(K1-P305), deletion of | ΔA-chain | KLSCGVKNRMHIRRKRIVGGKRAQLGDLPWQVA |
| A-chain of wt hCFI | (CFI-SPD) | IKDASGITCGGIYIGGCWILTAAHCLRASKTHRYQI |
| | | WTTVVDWIHPDLKRIVIEYVDRIIFHENYNAGTYQ |
| | | NDIALIEMKKDGNKKDCELPRSIPACVPWSPYLFQ |
| | | PNDTCIVSGWGREKDNERVFSLQWGEVKLISNCS |
| | | KFYGNRFYEKEMECAGTYDGSIDACKGDSGGPL |
| | | VCMDANNVTYVWGVVSWGENCGKPEFPGVYTK |
| | | VANYFDWISYHVGRPFISQYNV (SEQ ID NO: 12) |
| Wild type mouse CFI | Mouse CFI | MKLAHLSLFLLALHLSSSRSPSASDLPQEELVDQK |
| (https://www.uniprot.org/ | (mCFI) | CLLQKYTHRSCNKVFCQPWQRCIEGTCICKLPYQ |
| uniprot/Q61129) | | CPRAGTPVCAMNGRSYPTYCHQKSFECLHPEIKFS |
| | | HNGTCAAEGKFNVSLIYGRTKTEGLVQVKLVDQ |
| | | DERMFICKNSWSMAEANVACVDLGFPLGVRDIQ |
| | | GSFNISGNLHINDTECLHVHCRGVETSLAECAFTK |
| | | RRTELSNGLAGVVCYKQDADFPTSLSFQCVNGKH |
| | | IPQEKACNGVNDCGDQSDELCCKGCRGNASLCKS |
| | | GVCIPDQYKCNGEVDCITGEDESRCEEDRQQNIPK |
| | | GLARSAQGEAEIETEETEMLTPGMDNERKRIKSLL |
| | | PKLSCGVKRNTHTRRKRVIGGKPANVGDYPWQV |
| | | AIKDGQRITCGGIYIGGCWILTAAHCVRPSRAHSY |
| | | QVWTALLDWLKPNSQLGIQTVKRVIVHEKYNGA |
| | | TFQNDIALIEMKMHTGKKECELPNSVPACVPWSP |
| | | YLFQPNDRCIISGWGRGKDNQKVYSLRWGEVDLI |
| | | GNCSQFYPDRYYEKEMQCAGTRDGSIDACKGDS |
| | | GGPLVCEDINNVTYVWGIVSWGENCGKPEFPGVY |
| | | TRVANYFDWISYHVGRSLVSQHNV (SEQ ID NO: |
| | | 23) |
| wt hCFI + GSSGG | hCFI-hCFI | KVTYTSQEDLVEKKCLAKKYTHLSCDKVFCQPW |
| (linker) + wt hCFI | fusion | QRCIEGTCVCKLPYQCPKNGTAVCATNRRSFPTY |
| | | CQQKSLECLHPGTKFLNNGTCTAEGKFSVSLKHG |
| | | NTDSEGIVEVKLVDQDKTMFICKSSWSMREANVA |
| | | CLDLGFQQGADTQRRFKLSDLSINSTECLHVHCR |
| | | GLETSLAECTFTKRRTMGYQDFADVVCYTQKAD |
| | | SPMDDFFQCVNGKYISQMKACDGINDCGDQSDEL |
| | | CCKACQGKGFHCKSGVCIPSQYQCNGEVDCITGE |
| | | DEVGCAGFASVTQEETEILTADMDAERRRIKSLLP |
| | | KLSCGVKNRMHIRRKRIVGGKRAQLGDLPWQVA |
| | | IKDASGITCGGIYIGGCWILTAAHCLRASKTHRYQI |
| | | WTTVVDWIHPDLKRIVIEYVDRIIFHENYNAGTYQ |
| | | NDIALIEMKKDGNKKDCELPRSIPACVPWSPYLFQ |
| | | PNDTCIVSGWGREKDNERVFSLQWGEVKLISNCS |
| | | KFYGNRFYEKEMECAGTYDGSIDACKGDSGGPL |
| | | VCMDANNVTYVWGVVSWGENCGKPEFPGVYTK |
| | | VANYFDWISYHVGRPFISQYNVGSSGGKVTYTSQ |
| | | EDLVEKKCLAKKYTHLSCDKVFCQPWQRCIEGTC |
| | | VCKLPYQCPKNGTAVCATNRRSFPTYCQQKSLEC |
| | | LHPGTKFLNNGTCTAEGKFSVSLKHGNTDSEGIVE |
| | | VKLVDQDKTMFICKSSWSMREANVACLDLGFQQ |
| | | GADTQRRFKLSDLSINSTECLHVHCRGLETSLAEC |
| | | TFTKRRTMGYQDFADVVCYTQKADSPMDDFFQC |
| | | VNGKYISQMKACDGINDCGDQSDELCCKACQGK |
| | | GFHCKSGVCIPSQYQCNGEVDCITGEDEVGCAGF |
| | | ASVTQEETEILTADMDAERRRIKSLLPKLSCGVKN |
| | | RMHIRRKRIVGGKRAQLGDLPWQVAIKDASGITC |
| | | GGIYIGGCWILTAAHCLRASKTHRYQIWTTVVDW |
| | | IHPDLKRIVIEYVDRIIFHENYNAGTYQNDIALIEM |
| | | KKDGNKKDCELPRSIPACVPWSPYLFQPNDTCIVS |
| | | GWGREKDNERVFSLQWGEVKLISNCSKFYGNRF |
| | | YEKEMECAGTYDGSIDACKGDSGGPLVCMDANN |
| | | VTYVWGVVSWGENCGKPEFPGVYTKVANYFDW |
| | | ISYHVGRPFISQYNV (SEQ ID NO: 16) |

In some embodiments, a base molecule itself may be a CFI variant, for example in some embodiments, a CFI variant comprising only the serine protease domain (CFI-SPD) itself is a CFI variant. In some embodiments, the CFI variants are derived from any base molecule of Table 1, and comprise modifications to loops corresponding to the loops of an unmodified CFI. In some embodiments, the CFI variants are derived from any base molecule of Table 1, and comprise substitution mutations. In some embodiments, the CFI variants are derived from any base molecule of Table 1, and comprise a deletion of one or more domains of CFI. In some embodiments, the CFI variants are derived from any base molecule of Table 1, and comprise an inversion of the A-chain and the B-chain of the CFI.

In some embodiments, provided herein are CFI variants comprising at least one CFI domain, wherein the at least one CFI domain corresponds to those of a wild type CFI of any species. For example, the amino acid sequence of the at least one CFI domain may comprise the amino acid sequence derived from a wild type human CFI as set forth in SEQ ID NO: 5. The CFI variants provided herein comprising an amino acid sequence derived from SEQ ID NO: 5 may comprise one or more modifications with respect to the sequence set forth in SEQ ID NO: 5. For example, the one or more modifications may include a deletion of one or more amino acid residues, substitution mutations of one or more amino acid residues, an addition of one or more amino acid residues, the deletion of one or more domains of CFI, the substitution of one or more domains of CFI, or the addition of one or more domains of CFI.

In some embodiments, provided herein are CFI variants comprising at least one CFI domain of any species, wherein the at least one CFI domain comprises any one or more CFI domains selected from: a serine protease domain (SPD), a Factor I membrane attack complex (FIMAC) domain, a scavenger receptor cysteine-rich domain (SRCR), a low density lipoprotein receptor 1 (LDLr1), and low density lipoprotein receptor 2 (LDLr2) domains. In some embodiments, the any one or more CFI domains are that of a human CFI. In some embodiments, the any one or more CFI domains comprise an amino acid sequence derived from the sequence set forth in SEQ ID NO: 5.

In some embodiments, the CFI variants comprise all domains of a wild type CFI, i.e., each one of the SPD, the FIMAC domain, the SRCR domain, the LDLr1 domain, and the LDLr2 domain, and comprises a modification in any one or more of these domains with respect to the wild type CFI.

In some embodiments, the CFI variants do not comprise all of the domains corresponding to that of the wild type CFI. In some embodiments, the CFI variants comprise the SPD. In some embodiments, the CFI variants comprise only the SPD, wherein the A-chain of the CFI has been deleted, referred to herein as “CFI-SPD.” In some embodiments, the CFI-SPD comprises the amino acid sequence set forth in SEQ ID NO: 12 (as shown in Table 1), which is the SPD of a human CFI. In some embodiments, the CFI-SPD comprises no further modifications with respect to that of a wild type CFI SPD. In some embodiments, the CFI-SPD comprises one or more modifications with respect to that of a wild type CFI SPD. In some embodiments, the CFI-SPD comprises at least one modification with respect to the amino acid sequence set forth in SEQ ID NO: 12.

Exemplary variants of CFI are described in further detail below. Exemplary CFI variants comprise one or more substitutions of amino acid residues with respect to a CFI having the amino acid sequence set forth in SEQ ID NO: 5. For example, a CFI variant that includes substitutions at positions S499 and 1500 will have substitutions at positions S499 and 1500 in the amino acid sequence set forth in SEQ ID NO: 5.

### Tuning of Activity and Specificity

The activity and specificity of the CFI variants provided herein can be tuned (adjusted) for particular applications and therapeutic indications. For example, activity and specificity can be tuned by selection of C3b degraders, or C4b degraders, or degraders of both C3b and C4b. As referred to herein, protease activity for a substrate refers to the ability of a CFI variant of the disclosure to cleave its substrates, C4b and C3b. This can be expressed in a number of ways, for example as an increase in C4b degrader activity, protease activity towards C4b, C3b degrader activity, protease activity towards C3b, yield of cleavage products, and the like.

As used herein a C3b degrader is a CFI variant that is capable of cleaving C3b; likewise, a C4b degrader is a CFI variant that is capable of cleaving C4b. The use of C3b degrader does not imply that it does not degrade C4b. A CFI variant can be both a C3b degrader, and a C4b degrader, and may, but not necessarily, show specificity for one over the other.

The CFI variants provided herein have modified characteristics that include increases or decreases in protease activity for a substrate as well increases or decreases in substrate specificity.

In some embodiments, the CFI variants of the disclosure that are specific C3b degraders are useful for the treatment of diseases.

In some embodiments, the CFI variants of the disclosure that are specific C4b degraders are useful for the treatment of diseases.

In some embodiments, the CFI variants of the disclosure that are both C4b and C3b degraders, and show an improved characteristic as compared to wild type CFI (e.g. increased activity for both C4b and C3b) are useful for the treatment of diseases.

### Exemplary CFI Variants

Provided herein are CFI variants comprising or consisting of at least one modification with respect to a wild type CFI.

Without limitation, the disclosure contemplates the exemplary CFI variants described in Table 2. The variants of Table 2 include modified CFIs, described herein. For avoidance of doubt, unless otherwise indicated, where a residue number is indicated, it refers to SEQ ID NO: 5 (wild type human CFI), or a sequence corresponding thereto. For avoidance of doubt, by way of example a variant whose description is P433A is a CFI variant comprising a P433A substitution, e.g. a CFI variant comprising a P433A substitution in SEQ ID NO: 5 (or a sequence corresponding thereto); the disclosure also provides for a CFI variant consisting of a P433A substitution, e.g. a CFI variant, wherein SEQ ID NO: 5 has a P433A substitution.

CFI variants of the disclosure may have at least one, at least two, at least three, at least four, at least five, at least six, at least seven, or more modifications, e.g. substitutions, deletions, insertions and fusions. Modification, e.g. substitutions, for a given variant may be represented in one of many ways recognized by the skilled artisan. For example, a hCFI variant having substitutions at D395A and E416A may be referred to as having substitutions: “D395A and E416A”, “D395A-E416A”, “D395A+E416A”, “D395A/E416A”, or “D395A; E416A” and are used interchangeably herein. In some instances, a CFI variant having substitutions at D395A and E416A may be referred to as “hCFI; D39SA; E416A” or CFI variant (D395A; E416A).” As described herein, variants with other modifications, such as deletions, or combinations of modifications, such as deletions, fusions and substitutions, can conform to similar styles of nomenclature.

Table 2 provides exemplary CFI variants of the disclosure. This and other tables disclosing variants may include the following symbols and abbreviations and associated meanings: HSA=human serum albumin; CFI=complement factor I; Δ=Deletion of the amino acid range noted; → or >=Deletion of noted sequence and replaced with noted amino acids

TABLE 2
Exemplary CFI Variants and CFI Variant Combinations

| Variant Description | Variant Description | Variant Description |
|---|---|---|
| K14A | ΔC-term (Δ558- | N531A |
| Y20A | PFISQYNV-565 | P535A |
| Y20F | (SEQ ID NO: 25)) | Y408F |
| D26A | R557A | Y408F; N531G |
| F29A | K326A; R327A | Y408L; N531G; E457G; |
| R35A | Y408L; N531G | E461Q; R462K; F464Y |
| E38A | L307G | E530D |
| M220A; K221Q | N531G; P535A | E457G |
| S507A | Y408L | E461Q |
| S250A | 456-REKDNERVFS-465 | R462K |
| S250L | (SEQ ID NO: 26) → | F464Y |
| Δ(K1-P305) | NTASSGADYPDE | I317D; R318D; R319D; |
| D425A | (SEQ ID NO: 27) | K320D; R321K |
| D425K | E457G; E461Q; R462K; | Δ(K1-P305); N531G |
| D425R | F464Y | Δ(K1-P305); Y408L; |
| 514-MDANNVT-520 | E38A; D425R | N531G |
| (SEQ ID NO: 24) → NG | Y20F; D425R | Δ(KI-P305); N531G; |
| Y408L; N531G; E457G | S250A; D425R | P535A |
| Y408L; N531G; E457G; | NS31G | P535G |
| E461Q | F537K | V311-V565-G(13) |
| Δ(K1-P305); Y408L; | F537R | (SEQ ID NO: 29)- |
| N531G; E457G; E461Q; | Q467K | K1-G310 |
| R462K; F464Y | Q467R | C309S |
| Y408L; N531G; P535A | Q467K; F537K | C435S |
| Δ(K1-P305); | E530G | Y408L; N531G; E457G; |
| I317D; R318D; R319D; | E530G; N531G | E461Q; R462K |
| K320D; R321K | E530F | Y408L; N531G; E457G; |
| K14A; D425R | E530Y | E461Q: F464Y |
| Y408G | E530D; F537K | Y408L; N531G; E457G; |
| Y408P | R557K | R462K: F464Y |
| Y408D | P558L | Y408L; N531G; E461Q: |
| Y408A | E457G; E461Q | R462K; F464Y |
| Y408N | R462A | Y408L; E457G; E461Q; |
| Y408T | R462D | R462K: F464Y |
| Y408K | E457G; E461G | E457G; N531G; E461Q; |
| Y408R | N531G; E457G; E461Q | R462K; F464Y |
| Y408H | W381K | Y408L; E457G; E461Q; |
| Y408I | N404G | R462K |
| P535K | DS06A | N531G; E457G; E461Q; |
| K534Q | D506V | F464Y |
| E530D; N531G; G533A; | D506E | E416A |
| K534Q; P535K; E536N | D506G | Y408L; N531G; E457G; |
| R321A | I322V | E461Q; R462K; F464Y; |
| N402E | I322V; V323I | S507A |
| N422K | R327P | H370A |
| A502S; K504Q; F537K | I322V; V323I; R327P | P384A |
| A502S | V323A | P384G |
| K504Q | A328C; W468C | 420-DGNK-424 |
| K504E | A328C; W468C; K326Y; | (SEQ ID NO: 30) → GG |
| K504R | R327N | E536A |
| K504A | Y408L; N531G; E461Q | N85Q |
| K504G | D425R; Y408L; N531G; | N159Q |
| K504L | E457G; E461Q; R462K; | N476Q |
| K504P | F464Y | N518Q |
| K504H | Y20F; E38A; S250A; | N52Q; N85Q; N159Q |
| A361G | D425A | N446Q; N476Q; N518Q |
| T495F; Y496L; D497E; | Y20F; E38A; S250A; | B457A |
| S499G; I500K | D425A; Y408L; N531G; | E457D |
| T495F; Y496L; D497E; | E457G; E461Q; R462K; | E457F |
| S499G; I500K; G533A; | F464Y | E457H |
| K534Q; P535K; E536N; | V311-V565 | E457I |
| F537K | K1-G310 | E457K |
| E457Q | F559L | E457L |
| E457R | I560V | E457M |
| E457S | Y563H | E457N |
| E457T | P558S; F559L; I560V; | E457P |
| E457W | Y563H | K458A |
| E457Y | P558G | G469L; R456N; E457T; |
| E457V | L304G; P305G; K306G; | K458A |
| Y408E | L307G; S308G | G469L; R456N; |
| K14A; Y20F; D26A; | N531D | K458A |
| R35A; E38A | N531E | G469L; R456N; K458A; |
| K14A; Y20F; D26A; | NS31F | E461G |
| R35A; E38A; L304G; | NS31H | G469L; R456N; K458A; |
| P305G; K306G; L307G; | NS31I | E461G; F537K |
| S308G | NS31K | K504D |
| Y408M | N531L | K504F |
| Y408Q | N531M | K504I |
| Y408S | 1322T | K504M |
| Y408W | N531P | K504N |
| D341A | N531Q | K504S |
| Y408V | N531R | K504T |
| E461A | N531S | K504V |
| E461D | N531T | K504W |
| E461F | N531V | K504Y |
| E461G | N531W | G406D |
| E461H | N531Y | G406E |
| E461I | Y403F | G406F |
| E461L | A405S | G406H |
| E461M | G406R | G406I |
| E461N | Q409D | G406K |
| E461P | A405S; G406R; Y408L; | G406L |
| E461S | Q409D | G406M |
| E461T | A405S; G406A; Y408L; | G406N |
| E461W | Q409D | G406P |
| E461Y | Q409Y | G406Q |
| E461V | Q409H | G406S |
| R456A | G406A | G406T |
| I317D-R318D-R319D- | G406A; Y408L | G406V |
| K320D-R321K; Y408L; | T377G | G406W |
| N531G; E457G; E461Q; | W381A | G406Y |
| R462K; F464Y | W381A; P384A | G406D; Y408L |
| K312A | W381A; ΔP384 | G406D; N531G |
| R314A | G469L | G406D; P535A |
| K312A; R314A | R456N | G406D; Y408L; N531G |
| P558S | Y408L; N531G; E457G; | G406D; Y408L; P535A |
| I345G | E461Q; R462K; F464Y; | G406D; N531G; P535A |
| K340G; I345G | S507A | G406D; Y408L; N531G; |
| Y372G | E457G; S507A | P535A |
| P384A | N531G; P535A; S507A | K340G |
| P384G | F208Y | N531G; E457G; R462K; |
| W381G | F246Y | F464Y |
| V390G | F480Y | N531G; |
| W381G; V390G | F537Y | E461Q; R462K; |
| W381G; P384A; V390G | F208Y; F246Y; F480Y; | F464Y |
| W381G; P384G; V390G | F537Y | Y408L; N531G; R462K |
| N404G | H362T; V463S; R456I; | Y408L; N531G; F464Y |
| Q409G | D459W; S343R | Y408L; E457G; E461Q |
| K418G | H362T; V463S; R456I; | Y408L; E457G; R462K |
| D425G | D459W; S343K | Y408L; E457G; F464Y |
| K418G; D425G | H362T; V463S; R456F; | Y408L; E461Q; R462K |
| S465G | D459W; S343R | Y408L; E461Q; F464Y |
| G344R | H362T; V463S; R456I; | Y408L; R462K; F464Y |
| G344K | S343R | N531G; E457G; R462K |
| G344Y | H362T; R456I; D459W; | N531G; E457G; F464Y |
| T346R | S343R | N531G; E461Q; R462K |
| T346K | H362T; R456I; | N531G; E461Q; F464Y |
| T346H | S343R | N531G; R462K; F464Y |
| K504E | H362T; R456I; S343K | E457G; E461Q; R462K |
| K504D | K14A; D425R; Y408L- | E457G; E461Q; F464Y |
| E530R | N531G | E457G; R462K; F464Y |
| E530K | Y408L; E457G; S507A; | E461Q; R462K; F464Y |
| T346R; K504E; E530R | N531G | Y408L; N531G |
| T346K; K504D; E530K | E457G; N531G | Y408L; E461Q |
| G344R; Y408L; N531G | E457G; Y408L | Y408L; R462K |
| G344K; Y408L; N531G | Y408L; N531G; B457G; | Y408L; F464Y |
| T346R; Y408L; N531G | R462K | N531G; E461Q |
| T346K; Y408L; N531G | Y408L; N531G; E457G; | N531G; R462K |
| K504D; Y408L; N531G | F464Y | N531G; F464Y |
| K504E; Y408L; N531G | Y408L; N531G; E461Q; | E457G; R462K |
| Y408L; E530R; N531G | R462K | E457G; F464Y |
| Y408L; E530K; N531G | Y408L; N531G; E461Q; | E461Q; R462K |
| T346R; Y408L; K504E; | F464Y | E461Q: F464Y |
| E530R; N531G | Y408L; N531G; R462K; | R462K; F464Y |
| T346K; Y408L; K504D; | F464Y | Y408L; N422K |
| E530K; NS31G | Y408L; E457G; E461Q; | E457G; N422K |
| Y408L; S507A; NS31G | F464Y | N531G; N422K |
| E457G; N531G; N422K | Y408L; E457G; R462K; | P535G; N422K |
| Y408L; E457G; N531G; | F464Y | Y408L; P535G; N422K |
| N422K | Y408L; E461Q; R462K; | E457G; |
| Y408L; E457G; P535G; | F464Y | P535G; N422K |
| N422K | N531G; E457G; E461Q; | N531G; P535G; N422K |
| E457G; N531G; P535G; | R462K | Y408L; E457G; N422K |
| N422K | Y408L; N531G; D425R; | Y408L; N531G; N422K |
| Y408L; E457G; N531G; | E530Y | K424N |
| P535G; N422K | E457G; N531G; D425R; | K424Q |
| Y408L; E416A | E530Y | K424R |
| E457G; E416A | Y408L; E457G; NS31G; | K424H |
| N531G; E416A | D425R; E530Y | K424D |
| P535G; E416A | D425R; Y408L; N531G; | K424E |
| Y408L; D425R; E416A | E457G; E461Q; R462K; | K423G |
| E457G; D425R; E416A | F464Y; E530Y | K423A |
| N531G; D425R; E416A | E457G; N531G; E461Q; | K423E |
| Y408L; E457G; E416A | R462K; F464Y; E530Y | K423D |
| Y408L; NS31G; E416A | R365A | D549A |
| E457G; NS31G; E416A | R365V | D549V |
| Y408L; E457G; N531G; | R365I | D549L |
| E416A | R365L | D549M |
| Y408L; E457G; D425R; | R365M | D549F |
| E416A | R365F | D549Y |
| Y408L; N531G; D425R; | R365Y | D549W |
| E416A | R365W | D549T |
| E457G; N531G; D425R: | R365G | D549N |
| E416A | R365P | D549Q |
| D425R; Y408L; N531G; | R365S | D549G |
| E457G; E461Q; R462K; | R365T | D549P |
| F464Y; E416A | R365N | D549R |
| E457G; N531G; E461Q; | R365Q | D549H |
| R462K; F464Y; E416A | R365H | D549K |
| Y408L; E530Y | R365K | Y553A |
| E457G; E530Y | R365D | Y553V |
| N531G; E530Y | R365E | Y553I |
| P535G; E530Y | A366G | Y553L |
| Y408L; D425R; E530Y | K368G | Y553S |
| E457G; D425R; E530Y | K368E | Y553N |
| N531G; D425R; E530Y | K424A | Y553Q |
| Y408L; E457G; E530Y | K424V | Y553R |
| Y408L; N531G; E530Y | K424I | Y553H |
| E457G; N531G; E530Y | K424L | Y553K |
| Y408L: E457G; N531G; | K424M | Y553E |
| E530Y | K424F | R557V |
| Y408L; E457G; D425R; | K424Y | R557I |
| E530Y | K424W | R557L |
| R557W | K424G | R557M |
| R557S | K424P | R557F |
| R557T | K424S | R557Y |
| R557N | K424T | Y408F; E457G; E461Q; |
| R557Q | Q69G | N531G |
| R557G | L73G | Y408F; E457G; E461Q; |
| R557P | L76G | R462K; F464Y; N531G |
| R557H | H362G | Y408F; E457G; E461Q; |
| R557D | H370G | R462K; N531G |
| R557E | F399G | Y408F: E457G; E461Q; |
| T377G; N531G | E401G | F464Y; N531G |
| T377G; E457G | A405G | E457G; E461Q; R462K; |
| T377G; E461Q | R456G | F464Y; N531G; R557K |
| T377G; E457G; E461Q | D459G | B457G; E461Q; F464Y; |
| T377G; E457G; E461Q; | R484G | N531G; R557K |
| N531G | D501G | E530F; P558S |
| Y408L; N531G; R557A | A502G | E530Y: P558S |
| N531G; P535A; R557A | V526G | E457G; E461Q; E530F; |
| E457G; E461Q; R557A | S527G | N531G: P558S |
| N531G; E457G; E461Q; | W528G | E457G; E461Q; R462K; |
| R557A | F537G | F464Y; E530F; N531G; |
| Y408L; E457G; E461Q; | P538G | P558S |
| R462K; N531G; R557A | V540G | Y408L; E457G; E461Q; |
| N531G; P535A; R557K | Y553G | R462K; E530F; N531G; |
| E457G; E461Q; R557K | A342G | P558S |
| N531G; B457G; E461Q; | R371G | B457G; E461Q; F464Y; |
| R557K | R327G | E530F; N531G; P558S |
| Y408L; E457G; E461Q; | S343G | E457G; E461Q; E530Y; |
| R462K; N531G; R557K | Q373G | N531G; P558S |
| Y408L; N531G; ΔC-term | W375G | E457G; E461Q; R462K; |
| (Δ558-PFISQYNV-565 | I382G | F464Y; E530Y; N531G; |
| (SEQ ID NO: 25)) | H383G | P558S |
| N531G; P535A; ΔC-term | L386G | Y408L; E457G; E461Q; |
| (Δ558-PFISQYNV-565 | K387G | R462K; E530Y; N531G; |
| (SEQ ID NO: 25)) | R388G | P5588 |
| N531G; E457G; E461Q; | I389G | Y408F; E457G; E461Q; |
| ΔC-term (Δ558- | I391G | R462K; E530Y; N531G; |
| PFISQYNV-565 | E392G | P558S |
| (SEQ ID NO: 25) | Y393G | E457G; E461Q; F464Y; |
| Y408L; E457G; E461Q; | K419G | E530Y; N531G; P558S |
| R462K; N531G; ΔC-term | D420G | E457G; E461Q; K504H; |
| (A558-PFISQYNV-565 | N422G | N531G |
| (SEQ ID NO: 25)) | N460G | E457G; E461Q; R462K; |
| ΔC-term (4557- | R462G | F464Y; K504H; N531G |
| RPFISQYNV-565 | V463G | Y408L; E457G; E461Q; |
| (SEQ ID NO: 28)) | D425R; P558S; E457G; | R462K; K504H; N531G |
| Y408F; E416A; E457G; | E461Q; N531G | E457G; E461Q; F464Y; |
| E461Q; R462K; N531G | D425R; E530F; E457G; | K504H: N531G |
| E416A; E457G; E461Q; | E461Q; N531G | B416A; E457G; E461Q; |
| F464Y; N531G | D425K; E530F; E457G; | N531G |
| T377G; E457G; E461Q; | E461Q; N531G | Y408L; E416A; E457G; |
| R462K; F464Y; N531G | D425R; E530F; P558G; | E461Q; R462K; N531G |
| T377G; Y408L; E457G; | E457G; E461Q; N531G | E457G; E461Q; N422K |
| E461Q; R462K; N531G | K504R; E530F; P558G; | E457G; N422K; T377G |
| T377G; E457G; E461Q; | E457G; E461Q; N531G | E461Q; N422K; T377G |
| F464Y; N531G | K504R; D425R; P558G; | N531G; E457G; N422K; |
| T377G; E416A; K504H | E457G; E461Q; N531G | T377G |
| E416A; K504H | K504R; D425R; E530F; | N531G; E461Q; N422K; |
| T377G; K504H | E457G; E461Q; N531G | T377G |
| N422K; E457G; E461Q; | R557A; N531M | E457G; |
| N531G | R557K; N531M | E461Q; N422K; |
| N422K; E457G; E461Q; | R557A; N531M; Y403F; | T377G |
| Q467K; N531G | K504Y | T377G; |
| E416A; N422K; E457G; | R557A; N531D; Y403F; | N422K; |
| E461Q; Q467K; N531G | K504Y | E457G; |
| K504R; E530F; D425K; | R557A; NS31M; Y403F; | E461Q; N531G |
| P558S | K504Y; E457G; E461Q | D425K; Y408M |
| K504R; E530F; D425R; | R557A; N531G; Y403F; | D425K; E530F |
| P558S | K504Y; E457G; E461Q | D425K; F537K |
| K504R; E530F; D425R; | R557A; NS31D; Y403F; | D425K; K504R |
| P558G | K504Y; E457G; E461Q | D425K; P558S |
| K504R; E530F; D425K; | R557A; N531M; Y403F; | Y408M; E530F |
| P558G | K504Y; E457G; E461L | Y408M; K504R |
| K504R; E530F; D425K; | R557A: N531M; Y403F; | Y408M; P558S |
| P558S; E457G; E461Q; | K504Y: E457G; E461T | E530F; F537K |
| N531G | R557A; N531M; Y403F; | E530F; K504R |
| K504R; E457G; E461Q; | K504Y; E457G; E461V | E530F; P558S |
| N531G | R557A; N531M; Y403F; | F537K; K504R |
| E530F; E457G; E461Q; | K504Y; E457N; E461Q | F537K; P558S |
| N531G | R557A; N531M; Y403F; | K504R; P558S |
| D425R; E457G; E461Q; | K504Y; E457N; E461L | D425K; Y408M; F537K |
| N531G | R557A; N531M; Y403F; | D425K; Y408M; K504R |
| D425K; E457G; E461Q; | K504Y; E457N; E461T | D425K; Y408M; E530F; |
| N531G | R557A; NS31M; Y403F; | F537K |
| P558S; E457G; E461Q; | K504Y; E457N; E461V | D425K; Y408M; E530F; |
| N531G | N531M; Y403F; K504Y; | P558S |
| P558G; E457G; E461Q; | E457G; E461Q | D425K; E530F; F537K; |
| N531G | N422K; E461Q | K504R |
| K504R; E530F; E457G; | T377G; N422K | Y408M; E530F; F537K; |
| E461Q; N531G | N531G; E457G; T377G | K504R |
| K504R; D425R; E457G; | N531G; E461Q; N422K | Y408M; F537K; K504R; |
| E461Q; N531G | N531G; E461Q; T377G | P558S |
| K504R; P558S; E457G; | N531G; N422K; T377G | D425K; Y408M; E530F; |
| E461Q; NS31G | Y403F; G406A; D425K; | F537K; K504R |
| E530F; P558S; E457G; | E457G; E461Q; K504Y; | D425K; Y408M; E530F; |
| E461Q; N531G | N531G | F537K; P558S |
| D425K; E530F; F537K; | D425K; E457G; E461Q; | D425K; Y408M; E530F; |
| K504R; P558S | N531G | K504R; P558S |
| D425K; Y408M; E530F; | D425K; E457G; E461Q; | D425K; Y408M; F537K; |
| F537K; K504R; P558S | N531G; R557A | K504R; P558S |
| D42SK; E457G; E461Q; | R557A | N402R |
| K504R; N531G | Δ(V565) | H554F |
| D425K; E457G; E461Q; | F559Y | H554K |
| N531G; P558S | Δ(S308) | R371E |
| T377G; Y408M; N422K; | F559W | A342R |
| E457G; E461Q; E530F; | F559H | K5IR; N52A; A55P; |
| N531G | L307F | T59M; R61G; F64Y |
| T377G; N422K; D425K; | L307H | N52A |
| E457G; E461Q; E530F; | L307W | A55P |
| N531G | L307Y | T59E |
| E457G; E461Q; N531G; | P433A | L73F |
| S507A | P433G | L73D |
| N531G; S507A | P433F | Δ(275-CAGFASVAQE- |
| E457G; S507A | Δ(Y496) | 284 (SEQ ID NO: 31)) > |
| E461Q; S507A | Δ(D497) | CEEDRQQNIPKGLARS |
| N422K; S507A | Δ(Y496; D497) | AQGEAEIETE |
| T377G; S507A | S499G | (SEQ ID NO: 32) |
| D425K; S507A | S499A | R484A |
| Y408M; S507A | S499K | R484K |
| P558S; S507A | S499V | R484E |
| E530F; S507A | Y496L | Δ(V565) > VG |
| F537K; S507A | S499G; I500K | Δ(V565) > VY |
| K504R; S507A | S499A; 1500K | E416K |
| Y408F; S507A | T495F; Y496L; S499G; | E416R |
| R557A; S507A | I500K | D395A |
| E416A; E457G; E461Q; | K423R | D395G |
| R462K; F464Y; N531G | R62A | D395K |
| N52Q; N159Q | R62E | D395R |
| N476Q; N518Q | R62L | D395A; D425A |
| Y408F; N531M | R62H | D395A; E416A |
| Y408F; K504Y | E401K | D395A; E416A; D425A |
| G406A; Y403F | E401R | N402D |
| D425K | E401A | K368D |
| Y403F; D425K; E457G; | E401F | K1103A |
| N531G | N402G | E1106A |
| G406A; D425K; E457G; | H948N | I1134G |
| E461Q; N531G | L950S | I208G |
| Y403F; G406A; D425K; | D988N | H383K; D385N; K387S; |
| E457G; E461Q; N531G | L990S | R388Q; E392Q; Y393T; |
| Y403F; D425K; E457G; | V1108N | D395K; K419M; D420H; |
| E461Q; K504Y; N531G | E1110S | G421T; N422G; D425E |
| Q69H; L73F; G79E; | | H383K; R388Q; E392Q; |
| T80I; L83S; N84H | | Y393T; K419M |
| Δ(1-KVTYTS-6 | | K51D; T59E; R61D |
| (SEQ ID NO: 33)) > | | Q113IN |
| RSPSASDLP | | I1133S |
| (SEQ ID NO: 34) | | N422A |
| M186E; G187L; Y188S; | | T377A |
| Q189N; D190G; F191L; | | |
| D193G | | |

### B. Fusion Constructs Comprising Complement Factor I

Provided herein are formulations for fusion constructs comprising a first component (CFI portion) comprising at least one domain of complement factor I, and at least a second component, wherein the first component and second and subsequent components are fused (e.g. contiguous or separated by an optional linker). These fusion constructs are referred to herein as “CFI fusion constructs” or simply as “fusion constructs.” In some embodiments, the fusion construct comprises additional components, e.g. a third component, a fourth component, etc.

In some embodiments, the second and subsequent components of the fusion construct is a protein. In some embodiments, the second and/or subsequent components is not a protein.

The components of the fusion constructs of the disclosure may be held together by optional linkers. They may be of any suitable length of at least one amino acid. A linker may be a flexible linker, and may be a peptide of about 1 to about 20 amino acid residues in length, wherein the amino acid residues may comprise glycine residues. The linker may also optionally comprise serine residues. Exemplary flexible linkers can include, but are not limited to, glycine polymers, glycine-serine polymers, glycine-alanine polymers, alanine-serine polymers, or any other suitable flexible linkers known in the art. An exemplary linker is (GGSS)ⁿ, wherein n is any number from about 1 to about 20 (SEQ ID NO: 35). An exemplary linker is (GGSS)ⁿGG (SEQ ID NO: 36), wherein n is any number from about 1 to about 20. An exemplary linker is (GGSSGG)ⁿ, wherein n is any number from about 1 to about 20 (SEQ ID NO: 37). In some embodiments, the linkers are protease-sensitive cleavable linkers. Exemplary linkers linking the fusion constructs can be 1-50, 5-50, 10-50, 15-50, 20-50, 25-50, 1-20, 2-20, 3-20, 4-20, 5-20, 6-20, 7-20, 8-20, 9-20, 10-20, 3-15, 3-10, 3-9, 3-8, 3-7, 3-6, 3-5, 4-15, 4-10, 4-9, 4-8, 4-7, 4-6, 4-5, 5-15, 5-10, 5-9, 5-8, 5-7, 5-6, 6-15, 6-10, 6-9, 6-8, or 6-7 amino acids in length.

### CFI+Half-Life Extender Fusion Constructs

In some embodiments, the fusion construct comprises a wild type CFI or CFI variant (first component), and at least a second component, and wherein the second component is a half-life extender. Because naturally occurring CFI has a relatively short half-life, it may be advantageous in some embodiments to increase the half-life of CFI or a variant thereof. By using a second component that is a half-life extender, the activity may increase, or it may improve another characteristic as compared to a wild type CFI. For example, a wild type CFI or a CFI variant may have their half-life extended by fusing the CFI to a half-life extender.

Exemplary half-life extenders include, but are not limited to albumin, such as human serum albumin, PEG, a non-biodegradable polymer, a biodegradable polymer, and Fc. In some embodiments, the second component is a protein, and is a half-life extender, such as albumin or Fc. In some embodiments, the second component is not a protein, and is a half-life extender, such as PEG. In some embodiments, the half-life extender is comprising peptide repeats.

In some embodiments, a second component is a half-life extender, and is albumin. It is noted that as used herein, albumin refers to any albumin such as any serum albumin, or an albumin variant, or albumin derivative. As an example, a variant of albumin includes any albumin comprising at least one modification corresponding to the amino acid sequence set forth in SEQ ID NO: 7 (wild type Human serum albumin (HSA)), or at least one modification corresponding to the amino acid sequence of an albumin of any non-human species. In exemplary embodiments, the albumin is human serum albumin (HSA) and is provided in SEQ ID NO: 7.

Exemplary fusion constructs comprising wild type CFI and HSA are referred to herein, as “CFI-HSA” and are discussed in further detail below. An exemplary fusion construct of the disclosure comprises the amino acid sequence of SEQ ID NO: 21.

In some embodiments, a fusion construct of the disclosure comprises albumin and a CFI variant of the disclosure. Exemplary CFI variants are provided in Table 2.

### Structural Arrangements of Fusion Constructs

In some embodiments, provided herein are fusion constructs comprising at least a first component, wherein the first component is any of the wild type CFI or CFI variants provided herein (CFI portion), and a second component, wherein the first component and second component are fused, and wherein the second component is fused to the N-terminal end of the CFI portion. In some embodiments, the second component is fused to the C-terminal end of the CFI portion. In some embodiments, the second component is fused to the C-terminal end of the CFI portion, and a third component is further fused to the N-terminal end of the CFI portion. In some embodiments, the second component is fused to the N-terminal end of the CFI portion, and a third component is further fused to the C-terminal end of the CFI portion.

Turning to Table 3, SEQ ID NO: 1 is the amino acid sequence of wild type plasma-derived human CFI, referred to as “CFI-PD”, and has a leader sequence. Wild type CFI used for fusion with a second component may comprise the amino acid sequence of SEQ ID NO: 5, which does not include the leader sequence present in SEQ ID NO: 1. A mouse Ig kappa chain V-III region MOPC 63 leader sequence (SEQ ID NO: 2) may instead be used for the recombinant production of any of the CFI fusion constructs provided herein. In some embodiments, provided herein are CFI fusion constructs comprising at least one CFI domain, wherein the at least one CFI domain comprises the amino acid sequence set forth in SEQ ID NO: 5.

An exemplary fusion construct of the disclosure comprises the amino acid sequence of SEQ ID NO: 21.

TABLE 3
Components of Exemplary CFI Fusion Constructs

| Description | Sequence |
|---|---|
| Wild type | MKLLHVFLLFLCFHLRFCKVTYTSQEDLVEKKCLAKKYTHLSCDKVFCQP |
| plasma-derived | WQRCIEGTCVCKLPYQCPKNGTAVCATNRRSFPTYCQQKSLECLHPGTKFL |
| human CFI (CFI- | NNGTCTAEGKFSVSLKHGNTDSEGIVEVKLVDQDKTMFICKSSWSMREAN |
| PD) | VACLDLGFQQGADTQRRFKLSDLSINSTECLHVHCRGLETSLAECTFTKRRT |
| | MGYQDFADVVCYTQKADSPMDDFFQCVNGKYISQMKACDGINDCGDQSD |
| | ELCCKACQGKGFHCKSGVCIPSQYQCNGEVDCITGEDEVGCAGFASVTQEE |
| | TEILTADMDAERRRIKSLLPKLSCGVKNRMHIRRKRIVGGKRAQLGDLPWQ |
| | VAIKDASGITCGGIYIGGCWILTAAHCLRASKTHRYQIWTTVVDWIHPDLK |
| | RIVIEYVDRIIFHENYNAGTYQNDIALIEMKKDGNKKDCELPRSIPACVPWSP |
| | YLFQPNDTCIVSGWGREKDNERVFSLQWGEVKLISNCSKFYGNRFYEKEM |
| | ECAGTYDGSIDACKGDSGGPLVCMDANNVTYVWGVVSWGENCGKPEFPG |
| | VYTKVANYFDWISYHVGRPFISQYNV (SEQ ID NO: 1) |
| Leader sequence | METDTLLLWVLLLWVPGSTG (SEQ ID NO: 2) |
| (mouse leader for | |
| CFI-HSA) | |
| Human CFI | MKLLHVFLLFLCFHLRFC (SEQ ID NO: 3) |
| leader sequence | |
| Wild type CFI of | KVTYTSQEDLVEKKCLAKKYTHLSCDKVFCQPWQRCIEGTCVCKLPYQCP |
| SEQ ID NO 1 | KNGTAVCATNRRSFPTYCQQKSLECLHPGTKFLNNGTCTAEGKFSVSLKHG |
| without signal | NTDSEGIVEVKLVDQDKTMFICKSSWSMREANVACLDLGFQQGADTQRRF |
| sequence | KLSDLSINSTECLHVHCRGLETSLAECTFTKRRTMGYQDFADVVCYTQKAD |
| | SPMDDFFQCVNGKYISQMKACDGINDCGDQSDELCCKACQGKGFHCKSGV |
| | CIPSQYQCNGEVDCITGEDEVGCAGFASVTQEETEILTADMDAERRRIKSLL |
| | PKLSCGVKNRMHIRRKRIVGGKRAQLGDLPWQVAIKDASGITCGGIYIGGC |
| | WILTAAHCLRASKTHRYQIWTTVVDWIHPDLKRIVIEYVDRIIFHENYNAGT |
| | YONDIALIEMKKDGNKKDCELPRSIPACVPWSPYLFQPNDTCIVSGWGREK |
| | DNERVFSLOWGEVKLISNCSKFYGNRFYEKEMECAGTYDGSIDACKGDSG |
| | GPLVCMDANNVTYVWGVVSWGENCGKPEFPGVYTKVANYFDWISYHVG |
| | RPFISQYNV (SEQ ID NO: 5) |
| Linker | GGSSGG (SEQ ID NO: 6) |
| Human serum | DAHKSEVAHRFKDLGEENFKALVLIAFAQYLOQCPFEDHVKLVNEVTEFA |
| albumin (HSA) | KTCVADESAENCDKSLHTLFGDKLCTVATLRETYGEMADCCAKQEPERNE |
| | CFLQHKDDNPNLPRLVRPEVDVMCTAFHDNEETFLKKYLYEIARRHPYFYA |
| | PELLFFAKRYKAAFTECCQAADKAACLLPKLDELRDEGKASSAKQRLKCA |
| | SLQKFGERAFKAWAVARLSQRFPKAEFAEVSKLVTDLTKVHTECCHGDLL |
| | ECADDRADLAKYICENQDSISSKLKECCEKPLLEKSHCIAEVENDEMPADLP |
| | SLAADFVESKDVCKNYAEAKDVFLGMFLYEYARRHPDYSVVLLLRLAKTY |
| | ETTLEKCCAAADPHECYAKVFDEFKPLVEEPQNLIKQNCELFEQLGEYKFQ |
| | NALLVRYTKKVPQVSTPTLVEVSRNLGKVGSKCCKHPEAKRMPCAEDYLS |
| | VVLNQLCVLHEKTPVSDRVTKCCTESLVNRRPCFSALEVDETYVPKEFNAE |
| | TFTFHADICTLSEKERQIKKQTALVELVKHKPKATKEQLKAVMDDFAAFVE |
| | KCCKADDKETCFAEEGKKLVAASQAALGL (SEQ ID NO: 7) |
| HSA linked with | DAHKSEVAHRFKDLGEENFKALVLIAFAQYLQQCPFEDHVKLVNEVTEFA |
| CFI (CFI-HSA) | KTCVADESAENCDKSLHTLFGDKLCTVATLRETYGEMADCCAKQEPERNE |
| | CFLQHKDDNPNLPRLVRPEVDVMCTAFHDNEETFLKKYLYEIARRHPYFYA |
| | PELLFFAKRYKAAFTECCQAADKAACLLPKLDELRDEGKASSAKQRLKCA |
| | SLQKFGERAFKAWAVARLSQRFPKAEFAEVSKLVTDLTKVHTECCHGDLL |
| | ECADDRADLAKYICENQDSISSKLKECCEKPLLEKSHCIAEVENDEMPADLP |
| | SLAADFVESKDVCKNYAEAKDVFLGMFLYEYARRHPDYSVVLLLRLAKTY |
| | ETTLEKCCAAADPHECYAKVFDEFKPLVEEPQNLIKQNCELFEQLGEYKFQ |
| | NALLVRYTKKVPQVSTPTLVEVSRNLGKVGSKCCKHPEAKRMPCAEDYLS |
| | VVLNQLCVLHEKTPVSDRVTKCCTESLVNRRPCFSALEVDETYVPKEFNAE |
| | TFTFHADICTLSEKERQIKKQTALVELVKHKPKATKEQLKAVMDDFAAFVE |
| | KCCKADDKETCFAEEGKKLVAASQAALGLGGSSGGKVTYTSQEDLVEKKC |
| | LAKKYTHLSCDKVFCQPWQRCIEGTCVCKLPYQCPKNGTAVCATNRRSFP |
| | TYCQQKSLECLHPGTKFLNNGTCTAEGKFSVSLKHGNTDSEGIVEVKLVDQ |
| | DKTMFICKSSWSMREANVACLDLGFQQGADTQRRFKLSDLSINSTECLHVH |
| | CRGLETSLAECTFTKRRTMGYQDFADVVCYTQKADSPMDDFFQCVNGKYI |
| | SQMKACDGINDCGDQSDELCCKACQGKGFHCKSGVCIPSQYQCNGEVDCI |
| | TGEDEVGCAGFASVTQEETEILTADMDAERRRIKSLLPKLSCGVKNRMHIR |
| | RKRIVGGKRAQLGDLPWQVAIKDASGITCGGIYIGGCWILTAAHCLRASKT |
| | HRYQIWTTVVDWIHPDLKRIVIEYVDRIIFHENYNAGTYQNDIALIEMKKDG |
| | NKKDCELPRSIPACVPWSPYLFQPNDTCIVSGWGREKDNERVFSLOWGEVK |
| | LISNCSKFYGNRFYEKEMECAGTYDGSIDACKGDSGGPLVCMDANNVTYV |
| | WGVVSWGENCGKPEFPGVYTKVANYFDWISYHVGRPFISQYNV (SEQ ID |
| | NO: 21) |

### III. Uses of CFI-Containing Formulations

The formulations of the disclosure may be used for therapeutics in a subject. As used herein, a subject includes any mammalian subject and includes primates, rodents, domestic animals, zoo animals, and pets. In some embodiments, the mammalian subject is a human subject. In some embodiments, the mammalian subject is a non-human primate.

### A. Treatment of Non-Ocular Conditions

In some embodiments, the formulations provided herein are useful for treating a non-ocular condition in a subject. In some embodiments, provided herein is a method of treating an ocular condition in a subject in need thereof, the method comprising administering to the subject a therapeutically effective amount of any one of the formulations provided herein.

In some embodiments, the non-ocular condition is characterized by a deficiency of CFI. In some embodiments, the non-ocular condition is characterized by dysregulation of the complement system.

In some embodiments, the non-ocular condition is a systemic acute indication. In some embodiments, the non-ocular condition is a systemic acute indication selected from the group consisting of: acute glomerulonephritis, acute renal injury, acute respiratory distress syndrome, bacterial meningitis, brain hemorrhage, burns, coronavirus infection, Epstein-Barr virus infection, hematopoietic stem cell transplantation, ischemia reperfusion injury, Lyme disease, myocardial infarction, organ transplantation, periodontitis, pneumonia, pre-eclampsia, schistosomiasis, sepsis, stroke, thromboembolism, and traumatic brain injury.

In some embodiments, the non-ocular condition is a systemic chronic indication. In some embodiments, the non-ocular condition is a systemic chronic indication selected from the group consisting of: Alzheimer's disease, anti-neutrophil cytoplasmic antibody (ANCA)-associated vasculitis, antiphospholipid syndrome, asthma, atherosclerosis, atypical hemolytic uremic syndrome (aHUS), autoimmune hemolytic anemia, bullous pemphigoid (BP), C3 glomerulopathy, chronic kidney failure, chronic obstructive pulmonary disease (COPD), Cold agglutinin disease (CAD), Crohn's disease, diabetic neuropathy, generalized myasthenia gravis (gMG), Granulomatosis with Polyangiitis (GPA), Guillain-Barré Syndrome (GBS), hereditary angioedema (HAE), hidradenitis suppurativa (HS), IgA nephropathy (IgAN), lupus nephritis (LN), membranous glomerulonephritis (MN), microscopic polyangiitis (MPA), motor neuron disease, multifocal motor neuropathy (MMN), multiple sclerosis (MS), non-insulin dependent diabetes, osteoarthritis, pancreatitis, Parkinson's disease, paroxysmal nocturnal hemoglobinuria (PNH), post-transplant lymphoproliferative disease, protein losing enteropathy, psoriasis, pyoderma gangrenosum, rheumatoid arthritis, schizophrenia (SZ), systemic lupus erythematosus (SLE), immune thrombocytopenia (ITP), and ulcerative colitis, Lampert-Eaton myasthenic syndrome (LEMS), CHAPLE syndrome (CD55 deficiency), thrombotic microangiography (TMA) and chronic inflammatory demyelinating polyneuropathy (CIDP), Huntington disease and ischemia reperfusion injuries.

In some embodiments, the diseases that may be treated by use of the formulations provided herein that comprise CFI variants or fusion constructs that are C4b degraders include, but are not limited to a non-ocular condition. In some embodiments, the non-ocular condition is a systemic chronic indication. In some embodiments, the non-ocular condition is a systemic chronic indication selected from the group consisting of: Alzheimer's disease, Amyotrophic lateral sclerosis (ALS), anti-neutrophil cytoplasmic antibody (ANCA)-associated vasculitis, antiphospholipid syndrome, asthma, atherosclerosis, atypical hemolytic uremic syndrome (aHUS), autoimmune hemolytic anemia, bullous pemphigoid (BP), C3 glomerulopathy, chronic kidney failure, chronic obstructive pulmonary disease (COPD), Cold agglutinin disease (CAD), Crohn's disease, diabetic neuropathy, generalized myasthenia gravis (gMG), Granulomatosis with Polyangiitis (GPA), Guillain-Barré Syndrome (GBS), hereditary angioedema (HAE), hidradenitis suppurativa (HS), IgA nephropathy, lupus nephritis (LN), membranous glomerulonephritis (MN), microscopic polyangiitis (MPA), motor neuron disease, multifocal motor neuropathy (MMN), multiple sclerosis (MS), non-insulin dependent diabetes, osteoarthritis, pancreatitis, Parkinson's disease, paroxysmal nocturnal hemoglobinuria (PNH), post-transplant lymphoproliferative disease, protein losing enteropathy, psoriasis, pyoderma gangrenosum, rheumatoid arthritis, schizophrenia (SZ), systemic lupus erythematosus (SLE), immune thrombocytopenia (ITP), warm Autoimmune hemolytic anemia (wAIHA), Immune-Complex Membranoproliferative Glomerulonephritis (IC-MPGN), and ulcerative colitis, Lampert-Eaton myasthenic syndrome (LEMS), CHAPLE syndrome (CD55 deficiency), thrombotic microangiography (TMA) and chronic inflammatory demyelinating polyneuropathy (CIDP), Huntington disease and ischemia reperfusion injuries.

In some embodiments, the non-ocular condition is non-oncological.

In some embodiments, the non-ocular condition is oncological. In some embodiments, the non-ocular condition is oncological, and is characterized by solid tumors, or by liquid tumors. In some embodiments, the non-ocular condition is characterized by solid tumors, and is selected from the group consisting of: colorectal tumors, hormone-refractory prostate cancer, melanoma, metastatic breast cancer, metastatic colorectal cancer, metastatic esophageal cancer, metastatic pancreas cancer, metastatic stomach cancer, nasopharyngeal carcinoma, non-small cell lung cancer, pancreas tumors, squamous cell carcinoma, and stomach tumors. In some embodiments, the non-ocular condition is characterized by liquid tumors, and is selected from the group consisting of: acute myelogenous leukemia, B-cell lymphoma, and Hodgkin's disease.

### B. Treatment of Ocular Conditions

In some embodiments, the formulations provided herein are useful for treating an ocular condition in a subject. In some embodiments, provided herein is a method of treating an ocular condition in a subject in need thereof, the method comprising administering to the subject a therapeutically effective amount of any of the formulations provided herein.

In some embodiments, the ocular condition is characterized by a deficiency of CFI. In some embodiments, the ocular condition is characterized by dysregulation of the complement system.

In some embodiments, the ocular condition is characterized by the presence of a dysfunctional CFI gene. In some embodiments, the ocular condition is characterized by dysregulation of the complement system and low CFI levels.

In some embodiments, the ocular condition selected from the group consisting of: diabetic macular edema (DME), diabetic retinopathy, dry age-related macular degeneration (AMD), glaucoma, keratoconjunctivitis, neuromyelitis optica spectrum disorder (NMOSD), open angle glaucoma, polypoidal choroidal vasculopathy, Stargardt Disease, uveitis, and vitreoretinopathy.

In some embodiments, wherein the ocular condition is non-oncological.

### C. Administration

The in vivo administration of the formulations described herein may be carried out intravenously or subcutaneously.

In exemplary embodiments, administration of the formulations described herein is a subcutaneous administration. In some embodiments, the subcutaneous administration is a daily, every other day, twice weekly, or weekly administration.

In some embodiments, administration of the formulations described herein is an intravenous administration.

As generally contemplated herein, the CFI variants or fusion constructs described herein are delivered in an activated two chain form. However, in some instances, inactive CFI variants or fusion constructs can be delivered in an inactive single chain form. In some embodiments, what is delivered comprises both single chain inactive and two chain active forms.

### D. Dosages

In some embodiments, any of the formulations described herein may be administered to a subject in need thereof in a dosage of about 0.05 mg/kg to about 10 mg/kg. In some embodiments, the dosage is about 1 mg/kg. In some embodiments, administration of the therapeutic CFI, variants or fusion constructs in the formulations described herein is a subcutaneous administration, at a dosage of about 0.05 mg/kg, about 0.1 mg/kg, about 0.5 mg/kg, about 1 mg/kg, about 1.5 mg/kg, about 2 mg/kg, about 2.5 mg/kg, about 3 mg/kg, about 3.5 mg/kg, about 4 mg/kg, about 4.5 mg/kg, about 5 mg/kg, about 5.5 mg/kg, about 6 mg/kg, about 6.5 mg/kg, about 7 mg/kg, about 7.5 mg/kg, about 8 mg/kg, about 8.5 mg/kg, about 9 mg/kg, about 9.5 mg/kg, or about 10 mg/kg. In some embodiments, administration of the therapeutic CFI, variants or fusion constructs in the formulations described herein is an intravenous administration, at a dosage of about 0.1 mg/kg, about 0.5 mg/kg, about 1 mg/kg, about 1.5 mg/kg, about 2 mg/kg, about 2.5 mg/kg, about 3 mg/kg, about 3.5 mg/kg, about 4 mg/kg, about 4.5 mg/kg, about 5 mg/kg, about 5.5 mg/kg, about 6 mg/kg, about 6.5 mg/kg, about 7 mg/kg, about 7.5 mg/kg, about 8 mg/kg, about 8.5 mg/kg, about 9 mg/kg, about 9.5 mg/kg, or about 10 mg/kg. In some embodiments, administration of the therapeutic CFI variants or fusion constructs described herein is daily administration, every other day administration, weekly administration, or twice weekly administration.

In some embodiments, the target level of the therapeutic CFI, variants or fusion constructs in the formulations provided herein in plasma may be about 0.1 μg/ml, about 0.5 μg/ml, about 1 μg/ml, about 1.5 μg/ml, about 2 μg/ml, about 2.5 μg/ml, about 3 μg/ml, about 3.5 μg/ml, about 4 μg/ml, about 4.5 μg/ml, 5 μg/ml, about 5.5 μg/ml, about 6 μg/ml, about 6.5 μg/ml, about 7 μg/ml, about 7.5 μg/ml, about 8 μg/ml, about 8.5 μg/ml, about 9 μg/ml, about 9.5 μg/ml, about 10 μg/ml, about 10.5 μg/ml, about 11 μg/ml, about 11.5 μg/ml, about 12 μg/ml, about 12.5 μg/ml, about 13 μg/ml, about 13.5 μg/ml, about 14 μg/ml, about 14.5 μg/ml, 15 μg/ml, about 15.5 μg/ml, about 16 μg/ml, about 16.5 μg/ml, about 17 μg/ml, about 17.5 μg/ml, about 18 μg/ml, about 18.5 μg/ml, about 19 μg/ml, about 19.5 μg/ml, about 20 μg/ml, about 20.5 μg/ml, about 21 μg/ml, about 21.5 μg/ml, about 22 μg/ml, about 22.5 μg/ml, about 23 μg/ml, about 23.5 μg/ml, about 24 μg/ml, about 24.5 μg/ml, 25 μg/ml, about 25.5 μg/ml, about 26 μg/ml, about 26.5 μg/ml, about 27 μg/ml, about 27.5 μg/ml, about 28 μg/ml, about 28.5 μg/ml, about 29 μg/ml, about 29.5 μg/ml, about 30 μg/ml. The target level may be about 10 μg/ml, about 25 μg/ml, about 50 μg/ml, about 100 μg/ml, about 150 μg/ml, about 200 μg/ml, about 250 μg/ml, or even about 300 μg/ml.

### EXAMPLES

### Example 1: Preparation of CFI, CFI Variants and Fusion Proteins

### Overview

The methods provided below are applicable for expression, purification, activation, and in vitro sialylation of wild type CFI, CFI variants, and fusion constructs comprising wild type CFI and CFI variants.

For Example 1, reference to CFI-HSA refers to human serum albumin fused to the N-terminal end of a human wild type CFI (SEQ ID NO: 21).

A wild type CFI-HSA protein is expressed in Chinese hamster ovary (CHO) cells, purified with anti-albumin affinity purification, activated with furin, and purified by sizing columns. The activated CFI-HSA protein was subjected to in vitro sialylation to increase the total sialylation of CFI-HSA. Finally, the sialylated protein was purified using anti-albumin affinity purification and polished by size-exclusion column chromatography.

### Expression

The CFI-HSA gene (SEQ ID NO: 21) was synthesized (ThermoFisher Scientific, Geneart, Regensburg, Germany), with the human serum albumin at the amino terminus of the CFI protein. The protein was made with the signal sequence of SEQ ID NO: 2, which was removed during expression. The amino terminal albumin tag was connected to the CFI gene through a linker (SEQ ID NO: 6). The gene of CFI-HSA was inserted into an expression vector (Lake Pharma, Hayward, CA) utilizing standard molecular biology techniques. The resulting plasmid DNA was transformed into E. coli. The transfected E. coli were grown in 200 ml of LB media for expression of plasmid DNA and harvested utilizing standard techniques. The plasmid DNA was run on an agarose gel for quality assessment and sequence confirmed before proceeding to transfection.

1.0 liter of suspension TunaCHO™ cells were seeded in a shake flask and were expanded using serum-free chemically defined medium. On the day of transfection, the expanded cells were seeded into a new flask with fresh medium. The plasmid DNA was transiently transfected into the CHO cells using Lipofectamine 2000 (ThermoFisher Scientific). The cells were maintained as a batch-fed culture until the end of the production run. The protein was expressed for 14 days at 37° C. at 125 RMP with 8% CO₂ concentration. Cells were centrifuged and supernatant was collected for purification of secreted CFI-HSA at the end of 14 days expression.

### Purification

The supernatant with expressed CFI-HSA protein was passed through a 10 ml gravity flow column of CaptureSelect™ human albumin affinity matrix (ThermoFisher Scientific). Column-bound protein was washed with 10 column volume of 20 mM sodium phosphate buffer. Bound CFI-HSA protein was eluted in two steps: first, with 3 column volume of 20 mM Tris-HCl, pH 7.0 buffer with and 2 M MgCl2, and second, with 3 column volume of 20 mM citric acid, pH 3.0. Elution from both steps 1 and 2 was collected in 5 ml fractions. Each fraction of the step 2 elution was neutralized with 10% of neutralization buffer (1.5 M tris-HCL pH 7.4). All fractions were analyzed by reducing and non-reducing SDS-PAGE electrophoresis and bands were visualized by SimplyBlue™ SafeStain (ThermoFisher Scientific). CFI-HSA runs as a 130 kDa band on a non-reducing gel and as 102 kDa and 28 kDa bands on a reducing gel. Fractions with maximum CFI-HSA concentration and purity were pooled for further processing.

### Furin Activation

CFI-HSA is expressed as an inactive, single chain precursor protein, and is activated by furin, another serine protease. Furin is an endoprotease that cleaves CFI at its conserved RRKR sequence (also referred to as the furin recognition sequence), resulting in a heavy and light chain connected by a disulfide bond. The furin-processed, mature, two-chain protein is the activated form of the CFI protein.

Cleavage of CFI-HSA for producing the protein in its activated form was performed by incubation of 4 μg of recombinant furin per mg of purified CFI-HSA in Tris-NaCl (tris buffered saline), 2.5 mM CaCl₂) and 0.5% CHAPS at 30° C. for 18 hours. The CFI-HSA protein concentration was maintained at 1.4 mg/ml. This results in more than 90% activation of the protein. The activated protein was separated from inactivated CFI-HSA, and other proteins by size-exclusion chromatography. Size exclusion chromatography (SEC) was performed using a HiLoad 16/600 Superdex 200 column (GE Healthcare Life Sciences) and phosphate buffer saline (PBS, 137 mM NaCl, 2.7 mM KCl, 10 mM Na₂HPO₄, 2 mM KH₂PO₄, pH 7.4) as the mobile phase. Collected fractions were analyzed by CE-SDS (LabChip GXII, Perkin Elmer). Fractions containing the target protein were pooled and analyzed by SE-UPLC.

### In Vitro Sialylation

The activated CFI-HSA protein was subject to in vitro sialylation. Briefly, the sialylation was carried out in a two-step enzymatic reaction. First, a galactosylation reaction of CFI-HSA was performed in a 200 μl volume utilizing a 1:200 molar ratio of galactosyltransferase (GalT1) enzyme and CFI-HSA in 10 mM UDP-Galactose, 5 mM MnCl₂, and 100 mM MES, pH 6.5 buffer. Galactosylated CFI-HSA was purified from the reaction mixture by CaptureSelect™ Human Albumin affinity chromatography, as described earlier. Next, the sialylation reaction was performed in a 250 μl volume utilizing a 1:50 molar ratio of enzyme alpha 2,6-sialyl transferase and purified CFI-HSA in 80 μM Alkaline phospahatase, 6.1 mM CMP-NANA, 10 mM ZnCl₂ and 200 mM MES buffer, pH 6.5 at 37° C. for 1 hour. The sialylated CFI-HSA protein was purified from the reaction mixture by CaptureSelect™ Human Albumin affinity chromatography. The extent and characteristics of the sialic acid chain on CFI-HSA was determined by utilizing an Agilent/Prozyme Analytical service, GS-SAP method for total sialic acid quantitation (Agilent GS48), and mass spectrophotometric (MS) analysis (Lake Pharma analytical service), described in further detail below.

Briefly, total sialic acid quantitation was performed by mixing 20 μl of each sample with 10 μl of release reagent in a 96 well plate. The reaction mixture was incubated for 2 hours at 80° C. The samples were cooled to room temperature and 10 μl of labeling reagent was added to each sample for a further incubation of 3 hours at 50° C. The samples were again cooled down to room temperature and 160 μl of de-ionized (dI) water was added to bring the total volume to 200 μl. 10 μl of sample was injected in the Agilent UHPLC Poroshell C18 column to run at a flow rate of 0.4 ml/minute at 30° C. in 4% methanol, 8% acetonitrile in water (Line A1) and 100% ACN (Line B1). The peaks were recorded at 373/448 nm wavelength. A standard curve of total peak area versus picomoles (pmol) of sialic acid was generated by running 1-2000 pmol of NANA (N-acetylneuraminic acid, Neu5Ac) supplied with the kit on the same column. Total sialic acid of each sample was quantitated by comparing the peak area of samples against the standard curve.

The mass spectrometric analysis was performed by a standard trypsin Q-TOF mass spectrometer. Briefly, all samples were treated, reduced and alkylated by DTT and iodoacetamide, followed by trypsin digestion. The digested samples were analyzed by Waters ACQUITY UPLC coupled to a Xevo G2-XS-QTOF mass spectrometer using a protein BEH C18 column

### Polishing

Purified CFI-HSA protein was subjected to size-exclusion chromatography (SEC) using a HiLoad 16/600 Superdex 200 column (GE Healthcare Life Sciences) and phosphate buffer saline as the mobile phase. Collected fractions were analyzed by CE-SDS (LabChip GXII, Perkin Elmer). Fractions containing the target protein were pooled, and the concentration was brought to 5 mg/ml, and the samples were flash frozen for storage at −80° C.

### Expression and Purification of CFI-HSA Variants

The DNA of CFI-HSA variants was generated either by synthesis or by site-directed mutagenesis utilizing standard techniques. The proteins were expressed in 250 ml of suspension in TunaCHO™ cells, as described herein with reference to wild type CFI-HSA protein, with the exception that the expression was done for 7 days instead of 14 days. After 7 days, the cells were centrifuged, and conditioned media was passed through a gravity flow column of CaptureSelect™ human albumin affinity matrix (ThermoFisher Scientific). Column-bound protein was washed with 10 column volume of 20 mM sodium phosphate buffer. Bound CFI-HSA protein was eluted with 3 column volume of 20 mM Tris-HCl, pH 7.0 buffer with and 2 M MgCl₂ in 5 ml fractions. CFI-HSA or its variants were buffer exchanged (either by dialysis or a spin concentrator) into 30 mM HEPES, 150 mM NaCl, 2.5 mM CaCl₂), pH 7.4. Recombinant human furin, at a molar ratio of 1:25 (furin: CFI-HSA), was added to CFI-HSA and the reaction mixture was incubated at 30° C. for 16 hours. Two micrograms of the activation mixture was run on a 9% SDS-PAGE gel to assess the activation efficiency. Generally, more than 80% activation was achieved.

### Example 2: Formulation Development Study I

For Example 2, reference to CFI-HSA refers to human serum albumin fused to the N-terminal end of wild type CFI (SEQ ID NO: 21). CFI-HSA was formulated at a concentration of about 150 mg/mL.

The study was designed to investigate the physical and chemical properties of CFI-HSA, in a range of liquid and lyophilized formulations to assess the conditions that provide optimal options for stability. For example, to enable subcutaneous administration, a high concentration and stability of a CFI containing composition would be desired.

The active pharmaceutical ingredient (API) examined in this study was CFI-HAS (SEQ ID NO: 21). The material used for this study included the following:
(1) Drug Substance: CFI-HSA, 153.4 mg/mL, F1 formulation
(2) The chemicals and materials used to formulate and analyze CFI-HSA were as follows, show in Table 2.1:

TABLE 2.1
Materials

| Chemicals | Vendor | Catalog # | CAS # | Grade |
|---|---|---|---|---|
| Acetic acid | EMD | AX0073-9 | 64-19-7 | |
| Sorbitol | EMD | 111597 | 50-70-4 | EP, BP, NF, JP |
| NaCl | EMD | 106404 | 7647-14-5 | EP |
| Succinic acid | Sigma Aldrich | 1623411 | 110-15-6 | USP |
| Arginine.HCl | Sigma Aldrich | A4599 | 1119-34-2 | EP, JP, USP |
| Sucrose | Pfanstiehl | S-124-2-MC | 57-50-1 | NF, EP, JP, ChP |
| Trehalose | Pfanstiehl | T-104-4 | 6138-23-4 | NF, EP, JP, ChP |
| Glycine | JT Baker | 0581-05 | 56-40-6 | USP |
| Polysorbate 80 | Croda | Super Refined ™ | 9005-65-6 | USP, JP, EP |

In this stability study, the stability of CFI-HSA was monitored in twelve (12) formulations containing various tonicity modifiers/bulking agents across an optimal range of pH. Table 2.2 provides the formulation matrix for the twelve tested.
(1) pH 5-7
(2) Buffers (also referred to interchangeably herein as buffering agents)
(3) Tonicity modifiers (sorbitol or trehalose for non-ionic buffering agents, and sodium chloride for ionic buffering agents)
(4) Bulking agents (trehalose or glycine)
(5) Surfactants

TABLE 2.2
Formulation Matrix

| Form | Form. | | | Tonicity Modifier/ | API | |
|---|---|---|---|---|---|---|
| No. | Type | Buffer (20 mM) | pH | Bulking Agent | (mg/m L) | Surfactant |
| 1 | Liquid | Sodium | 5 | 5% Sorbitol | 150 | 0.02% PS 80 |
| | | acetate | | | | |
| 2 | Liquid | Sodium | 5.5 | 5% Sorbitol | 150 | 0.02% PS 80 |
| | | acetate | | | | |
| 3 | Liquid | Sodium | 5.5 | 150 mM NaCl | 150 | 0.02% PS 80 |
| | | acetate | | | | |
| 4 | Liquid | Sodium | 5.5 | 70 mM arginine. HCl, | 150 | 0.02% PS 80 |
| | | acetate | | 2% sucrose, 60 mM | | |
| | | | | glycine | | |
| 5 | Liquid | Histidine HCl | 6 | 70 mM arginine.HCl, | 150 | 0.02% PS 80 |
| | | | | 2% sucrose, 60 mM | | |
| | | | | glycine | | |
| 6 | Liquid | Histidine HCl | 6 | 150 mM NaCl | 150 | 0.02% PS 80 |
| | Lyo | Sodium | 5 | 10% Trehalose | 150 | 0.02% PS 80 |
| | | succinate | | | | |
| 8 | Lyo | Sodium | | 5% Trebalose | 150 | 0.02% PS 80 |
| | | succinate | | 2.0% Glycine | | |
| 9 | Lyo | Histidine HCl | 6 | 10% Trehalose | 150 | 0.02% PS 80 |
| 10 | Lyo | Histidine HCl | 6 | 5% Trehalose, 2% | 150 | 0.02% PS 80 |
| | | | | Glycine | | |
| 11 | Lyo | Histidine HCl | | 10% Trehalose | 150 | 0.02% PS 80 |
| 12 | Lyo | Histidine HCl | 7 | 5% Trehalose, 2% | 150 | 0.02% PS 80 |
| | | | | Glycine | | |

*Form. = Formulation; API = active pharmaceutical ingredient, CFI-HSA; Lyo = lyophilized

### Lyophilized Formulation Preparations

For each formulation, CFI-HSA DS solution was loaded into a dialysis cassette (Slide-A-Lyzer® Dialysis Cassette, 10,000 MWCO). Formulations were dialyzed into their respective formulation buffers.

Formulations were sterile-filtered through a 0.2 μm PES membrane in an aseptic BSC and filled to 0.2 mL into 2 cc sterilized glass vials. Vials were then partially stoppered with lyo vent stoppers and were lyophilized using the parameters outlined in Table 2. Following lyophilization, the chamber was backfilled with nitrogen and vials were stoppered. Vials were then removed from and stoppered, crimped, and labeled. Additional vials were set aside and frozen prior to lyophilization for pre-lyophilization analysis alongside time zero.

### Liquid Formulation Preparations

For each formulation, CFI-HSA DS solution was loaded into a dialysis cassette (Slide-A-Lyzer® Dialysis Cassette, 10,000 MWCO). Formulations were dialyzed into their respective formulation buffers. Formulations were sterile-filtered through a 0.2 μm PES membrane in an aseptic BSC and filled to 0.2 mL into 2 cc sterilized glass vials. Vials were then stoppered, crimped, and labeled.

### Analytical Methods

The following analytical methods were employed.
(1) Visual Inspection: Visual inspection was performed under a white light source (13 W fluorescent tube) against black or white background. Digital photographs were acquired of all formulations at every time point.
(2) Concentration Measurement: The concentration of CFI-HSA was analyzed by A280 via SoloVPE using an E.C. of 1.041 mg/mL⁻¹×cm⁻¹.
(3) SE-HPLC: Size-exclusion HPLC chromatography was executed with the following parameters.
   HPLC: Agilent 1260 system
   Column: Agilent AdvanceBio SEC 300 Å, 4.6×300 mm, 2.7 μm, LC columnP/N: PL1580-5301
   Mobile Phase: 150 mM Sodium Phosphate, pH 6.9±0.1
   Diluent: DI water
   Sample Concentration: 0.5 mg/mL
   Flow Rate: 0.35 mL/min
   Run Time: 15 min
   Autosampler Temp.: 5° C.
   Column Temp.: 30° C.
   Absorbance Detection: 280 nm, 215 nm
   Injection Volume: 20 μL
   Injection load: 15 minutes
(4) FlowCAM: The FlowCAM particle imaging system combines optics, electronics, and fluidicsfor automated analysis of particles. The optical system is used to capture real-time images of the particles in the fluid as they pass through the flow cell. The imaging software provides the ability to assess particle size and morphology. All samples were analyzed and degassed for 30 minutes at 75 torr prior to analysis.
(5) Sub-Ambient DSC: Using a Pyris Diamond DSC with an Intercooler II, approximately 10 μL of each sample was frozen at −60° C. At a ramp rate of 10° C. min, the sample was warmed until thawed and the heat flow during the warming process was recorded.
(6) Fourier Transform Infrared Spectroscopy (FTIR): FTIR is a technique used to obtain an infrared spectrum of absorption of a gas, liquid, or solid. IBI uses an FTIR-660 Plus spectrometer which collects absorption of infrared radiation by the sample material versus wavenumber [cm⁻¹]. This data yields information on secondary structure of polypeptides.

### Surfactant Screening

The CFI-HSA drug substance was diluted to 15 mg/mL with water. The solution was then sterile-filtered through a 0.2 μm syringe filter and filled into 3 cc vials at a fill volume of 1.0 mL. Four vials each were then directly spiked with the stock solutions of either 1% PS20, 1% PS80, or 10% F-68 to reach final concentrations of 0.015% PS20, 0.015% PS80, or 0.15% F-68. Four of the filled vials were used as a surfactant-free control. A total of four vials per condition were prepared. The vials per condition were placed on a shaker and were agitated at 1000 rpm for four hours at ambient temperature. One (1) vial per condition was concurrently incubated at ambient temperature for four hours.

TABLE 2.3
Surfactant Screen Matrix

| | CFI-HSA |
|---|---|
| Surfactant | (mg/mL) |
| None | 15.3 |
| (No Surfactant) | |
| 0.015% PS20 | |
| 0.015% PS80 | |
| 0.15% F-68 | |

The stability of CFI-HSA with and without surfactants was studied following agitation for four hours (1000 RPM on an orbital shaker, room temperature); and static conditions at four hours room temperature).

Following agitation and static conditions, all samples appeared clear, colorless, and free of visible particulates, regardless of surfactant.

After four hours of agitation or static incubation, similar chromatographic profiles were observed for all samples with or without PS80, PS20, and F68. All samples also showed similar purity and total peak area (Table 2.4).

TABLE 2.4
Surfactant Screening SE-HPLC Results

| | Static Samples | | Agitated Sample 1 | | Agitated Sample 2 | |
|---|---|---|---|---|---|---|
| | Percent Total Area (%) | | Percent Total Area (%) | | Percent Total Area (%) | |
| Sample | HMW 1 | Main Peak | HMW 1 | Main Peak | HMW 1 | Main Peak |
| No surfactant | 0.2 | 99.8 | 0.6 | 99.4 | 0.5 | 99.5 |
| Polysorbate 80 | 0.2 | 99.8 | 0.5 | 99.5 | 0.5 | 99.5 |
| Polysorbate 20 | 0.2 | 99.8 | 0.6 | 99.4 | 0.4 | 99.6 |
| Poloxamer F68 | 0.2 | 99.8 | 0.3 | 99.7 | 0.2 | 99.8 |

FlowCam Analysis is show in Table 2.5. Samples without a surfactant shows significant increase in the particle counts. However, all surfactant-containing samples displayed relatively lower particle concentrations, regardless of surfactant.

TABLE 2.5
Surfactant Screening FlowCam Results

| | Surfactants | | | | | | | | | | | | | | | |
|---|---|---|---|---|---|---|---|---|---|---|---|---|---|---|---|---|
| | No surfactant | | | | PS80 (0.02%) | | | | PS20 (0.01%) | | | | Pluronic F68 (0.1%) | | | |
| | | Agitation | | | | Agitation | | | | Agitation | | | | Agitation | | |
| Particles/mL | Static | 1 | 2 | 3 | Static | 1 | 2 | 3 | Static | 1 | 2 | 3 | Static | 1 | 2 | 3 |
| >2 μm | 498 | 5111 | 6896 | 1516 | 648 | 409 | 1904 | 818 | 259 | 1504 | 3442 | 1335 | 80 | 2143 | 988 | 2232 |
| >5 μm | 90 | 1365 | 2173 | 608 | 279 | 109 | 509 | 120 | 110 | 578 | 978 | 309 | 30 | 668 | 265 | 578 |
| >10 μm | 50 | 259 | 239 | 150 | 10 | 35 | 70 | 20 | 20 | 90 | 190 | 70 | 10 | 90 | 49 | 60 |
| >25 μm | 0 | 10 | 10 | 0 | 0 | 7 | 10 | 0 | 0 | 10 | 50 | 0 | 0 | 20 | 25 | 0 |

Based on these results, it was determined that the 0.02% polysorbate 80, was one optimal choice for the next round of formulation optimization study.

### Formulation Optimization

The objective of the accelerated stability study was to assess the stability of both liquid and lyophilized formulations, containing CFI-HSA at 150 mg/mL. All formulations were filled at 0.2 mL fill volumes and F7-F12 were lyophilized. Pre-lyophilized samples were frozen at −70° C. for the duration of the lyophilization cycle and were thawed at time zero for analysis.

This study included the incubation of all formulation candidates at refrigerated (5° C.), stressed (25° C.), and accelerated (40° C.) storage temperatures. The temperature storage portion of the study was performed over a four-week period. Table 2.6 summarizes storage conditions that were used for formulation stability evaluation.

TABLE 2.6
Summary of Stability Study Stress Conditions

| Stress | Conditions | Time Point(s) |
|---|---|---|
| Temperature | 5° C.; 25° C.; 40° C. | 0, 1, 2, 4 weeks |
| (Liquid) | | |
| Temperature | 5° C.; 25° C.; 40° C. | |
| (Lyophilized) | | |
| Agitation | Vortex | 4 hours at 1000 RPM |

At time zero, vials were placed at appropriate temperatures following Table 2.6. Vials were pulled at each time point and analyzed.

At time zero, all pre-lyophilized formulations appeared clear, colorless, and free of visible particulates. Lyophilized formulations displayed good cakes. All liquid and reconstituted lyophilized formulations appeared clear, slightly yellowish tint, and free of visible particulates. Throughout the study up to the four-week time point, no significant changes were observed in the lyophilized cakes, regardless of storage temperature. Similarly, most liquid and reconstituted lyophilized formulations appeared clear and yellow tint, with no visible particulates at all temperatures.

At time zero, all formulations displayed concentrations near the target, ranging from 130 mg/mL to 160 mg/mL. Throughout the study up to the four-week time point, most formulations at 5° C., 25° C., and 40° C. displayed concentrations within experimental variability. Table 2.7 shows the stability concentration results.

TABLE 2.7
Stability Concentration Results

| | | Concentration (mg/mL) | | | | | | | | |
|---|---|---|---|---|---|---|---|---|---|---|
| Form | | | T = 1 week | | T = 2 week | | | T = 4 week | | |
| No. | | T = 0 | 25° C. | 40° C. | 5° C. | 25° C. | 40° C. | 5° C. | 25° C. | 40° C. |
| 1 | LIQUID | 150.49 | 150.25 | 148.81 | 144.89 | 145.86 | 147.50 | 148.1 | 143.0 | 142.8 |
| 2 | | 144.67 | 157.14 | 156.21 | 153.71 | 151.85 | 154.11 | 155.5 | 155.4 | 158.8 |
| 3 | | 143.57 | 149.07 | 145,49 | 117.26 | 116.40 | 109.87 | 117.7 | 118.0 | 110.3 |
| 4 | | 141.64 | 147.39 | 139.79 | 136.98 | 136.45 | 130.40 | 138.3 | 139.5 | 133.0 |
| 5 | | 147.23 | 151.12 | 147.23 | 130.47 | 133.93 | 125.85 | 130.3 | 132.3 | 118.1 |
| 6 | | 128.91 | 135.09 | 127.99 | 118.92 | 120.16 | 107.19 | 119.3 | 119.4 | 94.7 |
| 7 | LYOPH | 156.46 | 180.31 | 175,71 | 170.60 | 174.47 | 174.83 | 168.9 | 174,2 | 185.4 |
| 8 | | 158.47 | 160,32 | 150.62 | 142.27 | 151,29 | 152.59 | 153.6 | 153.1 | 151.9 |
| 9 | | 178.19 | 170.42 | 174,25 | 171.15 | 155.01 | 171.56 | 151.3 | 174,2 | 169.4 |
| 10 | | 143.10 | 141.45 | 146.56 | 147.61 | 146.88 | 147.56 | 145.5 | 146.9 | 149.4 |
| 11 | | 147.09 | 154,29 | 139.70 | 146.20 | 143,73 | 143.09 | 146.8 | 143.6 | 151.6 |
| 12 | | 128.43 | 127.70 | 126,12 | 128.12 | 127.46 | 127.72 | 127.6 | 127,9 | 128.5 |
| 13 | | 137.87 | N/A | 136.39 | N/A | N/A | 199.96 | N/A | 148.8 | 143.9 |

FTIR spectrum of CFI-HSA appeared similar to what was reported from albumin with strong α-helical signal at 1650-1660 cm-1. No major change in the FTIR spectrum was observed when CFI-HSA was lyophilized in the candidate formulations.

At time zero, all formulations including reconstituted lyophilized formulations displayed comparable chromatographic profiles with minor HMW (high molecular weight) and LMW (low molecular weight) peaks. (SE-HPLC results)

After 1 week storage at 25-40° C., all liquid formulations displayed increase in HMWS as well as LMW. The increase of LMW was much faster at lower pHs. All lyophilized formulations, however, remained much more stable, especially with no sign of increase of LMW. (SE-HPLC results)

After 2 week storage at 5, 25 and 40° C., all liquid formulations displayed increase in HMWS as well as LMW. The increase of LMW was much faster at lower pHs. All lyophilized formulations, however, remained much more stable, especially with no sign of increase of LMW. (SE-HPLC results)

General degradation process remained consistent at 4 week time point at 5, 25 and 40° C.: all liquid formulations displayed increase in HMWS as well as LMW. The increase of LMW was much faster at lower pHs. All lyophilized formulations, however, remained more stable, especially with no sign of increase of LMW. (SE-HPLC results)

The rates of degradation in each tested formulation over 4 week time storage at 40° C. were compared. Among all tested formulations, in this study, lyophilized formulations containing both trehalose and glycine as bulking agents performed best at pH range of 6-7.

### Findings

The stability of CFI-HSA at 150 mg/mL in various formulation conditions was examined in this example. The conditions investigated included liquid and lyophilized formulations containing various buffers (20 mM sodium acetate, sodium succinate, or histidine·HCl), tonicity modifiers (sodium chloride, sorbitol, or trehalose), bulking agents (trehalose or glycine), and across pH values ranging from 5.0 to 7.0. Formulations were examined under static storage conditions at refrigerated (5° C.), ambient (25° C.), and accelerated (40° C.) temperatures for up to four (4) weeks Over the course of the study, analysis by visual inspection, concentration, SE-HPLC, and FlowCAM was carried out. Additionally, lyophilized formulations were also analyzed by DSC and FTIR.

Initially, a small surfactant screen was performed using commonly used surfactant stabilizers. The study evaluated the benefit of surfactants in stabilization of CFI-HSA to degradation following exposure to agitation induced shear stresses. CFI-HSA drug substance at 15 mg/mL was spiked with surfactants 0.015% PS20, 0.015% PS80, and 0.15% F68. Comparison of surfactant-containing vials following the agitation stress by SE-HPLC showed comparable profiles for all samples regardless of agitation. However, MFI analysis demonstrated surfactant-free samples displayed substantially increased sub-visible particulate concentrations compared to those containing surfactant. This provided evidence that CFI-HSA was sensitive to shear stress and improved stability could be achieved with addition of surfactant in further studies. The current surfactant in the drug substance, 0.02% PS80, was selected for the accelerated stability study.

Following the preparation of the 12 candidate formulations, all samples appeared clear and free of visible particulates at time zero.

Throughout the entire study, no significant changes in visual appearance were observed in the lyophilized cakes-regardless of storage temperature. Similarly, most liquid and reconstituted lyophilized formulations appeared clear with signature yellow tint at all temperatures. Following four weeks, most formulations were near target concentration at all storage conditions.

FTIR results found no significant differences in spectra profiles between pre-lyophilized and lyophilized samples.

FlowCAM analysis saw relatively low subtracted subvisible particle concentrations for all samples, suggesting that precipitation or particle is not a critical factor for properly formulated CFI-HSA.

The accelerated study was under conditions of extreme pressure and may not be predictive. Nonetheless, analysis by SE-HPLC following four weeks at all temperatures saw significantly higher LMW in liquid formulations, especially low pH formulations like F1, F2, F3, and F4. Even at pH 6, F5 and F6 saw approximately 1% increase in LMW during storage for four weeks at 5° C. Additives that are effective in inhibiting the autolysis of CFI-HSA may be useful to include. The autolysis is stopped when CFI-HSA is lyophilized. No significant increase in the LMW was seen when the lyophilized formulations were stored for four weeks at 40° C. Among tested, formulations F8, F10, and F12 containing glycine as a bulking agent, an ionic bulking agent as compared to non-ionic trehalose, showed improved stability against the aggregation. In addition, the aggregation was slower as the pH increased from 5 to 7.

The key degradation products and stability indicating assays were identified as increases in HMW and LMW by SE-HPLC. Based on all the results generated in this study, one leading formulation for CFI-HSA at 150 mg/mL may be the lyophilized condition of 20 mM histidine, 5.0% trehalose, 2.0% glycine, and 0.02% polysorbate 80 at pH 6.0-7.0.

### Example 3: Formulation Development Study II

Additional formulations for the active ingredients described herein were developed in an indepenf study. To enable subcutaneous administration, high concentration and stability of a CFI containing composition is desirable to achieve.

For Example 3, reference to CFI-HSA refers to human serum albumin fused to the N-terminal end of wild type CFI (SEQ ID NO: 21). CFI-HSA was formulated at a concentration between 30-189 mg/mL.

### Biophysical Characterization and pH Stability Profile

A stock solution of CFI-HSA at 5 mg/mL was dialyzed to citrate phosphate buffer containing 135 mM sodium chloride, at pH 5, 5.5, 6, 6.5, 7, or 7.5. Following visual appearance to assess any potential precipitation, the samples were 0.2 micron filtered, adjusted to 2 mg/ml in the corresponding buffer and aseptically transferred to microtubes. Sets of samples were stored at either −70° C., 25° C., or 40° C. After 7 days, sets were analyzed by appearance (opalescence and particles), protein concentration (OD280), SEC, DSF, DLS, icIEF, CE-SDS, and S2288 chromogenic activity assay.

Following 7 days of storage at either 25° C. or 40° C., all samples were found to be visually clear with no visibly detectable particles at both temperatures and all pH levels tested. Protein concentration was stable in all samples (Table 3.1).

TABLE 3.1
CFI-HSA concentration after one week storage at 25° C. and 40° C.

| | | | Concentration | |
|---|---|---|---|---|
| | | | (mg/mL) | |
| | | | just after | Concentration |
| | | | dialysis | (mg/mL) |
| Sample | | | adjusted from | post-dialysis |
| Code | pH | Temperature | 5 to 2 mg/ml | at one week |
| Sample A | 5.0 | 25° C. | 2.0 | 1.95 |
| Sample A | 5.0 | 40° C. | 2.0 | 1.94 |
| Sample B | 5.5 | 25° C. | 2.0 | 2.0 |
| Sample B | 5.5 | 40° C. | 2.0 | 2.02 |
| Sample C | 6.0 | 25° C. | 2.0 | 1.99 |
| Sample C | 6.0 | 40° C. | 2.0 | 2.02 |
| Sample D | 6.5 | 25° C. | 2.0 | 1.98 |
| Sample D | 6.5 | 40° C. | 2.0 | 1.99 |
| Sample E | 7.0 | 25° C. | 2.0 | 1.97 |
| Sample E | 7.0 | 40° C. | 2.0 | 1.99 |
| Sample F | 7.5 | 25° C. | 2.0 | 2.02 |
| Sample F | 7.5 | 40° C. | 2.0 | 2.04 |

SEC-HPLC was used to assess aggregation under storage conditions. At 25° C. storage, the main peak purity showed a steady increase as the pH decreased from 7.5 to 5.0. On the contrary, at pH 5.0 and 40° C. storage conditions, the main peak purity had a sharp decrease and the lower molecular weight species had a spike, indicating potential protein degradation had occurred. In summary, under these conditions, a pH of between 5.5 and 6.0 appeared to be the optimal range for CFI-HSA stability.

The hydrodynamic size of the ensemble collection of particles was measured by dynamic light scattering (DLS) to further assess aggregation under storage conditions. The results showed that both the intensity weighted mean (Zave) and polydispersity increased steeply at pH 5, but were comparable between pH 6-7.5. Meanwhile, size of the major species (>99.8%) increased slightly with increasing pH. Taken together, the polydispersity and particle size data measured by DLS indicated an optimal storage pH of about 6, corroborating the SEC-HPLC results.

CE-SDS was performed to assess the purity changes using the pre-formulations. Results showed a small decrease in purity at pH 5 and 7.5 and comparable purity between pH 5.5-6.5 by both reducing and non-reducing CE-SDS. Similarly, optimum pH levels between 5.5 and 7.0 were suggested by differential scanning fluorimetry (DSF), while pH 5 was found to be the least stable by imaged capillary isoelectric focusing (icIEF).

### High Concentration Liquid Formulations

Feasibility of a high concentration formulation was assessed. Purified CFI-HSA was dialyzed to 20 mM histidine, 150 mM ArgHCl, pH of 5.8, and concentrated to 189 mg/mL in a spin filter unit. A high concentration of arginine was used to increase solubility and prevent excessive viscosity caused by the concentrated CFI-HSA. To evaluate the impact of protein concentration and arginine concentration on viscosity, concentrated sample at 189 mg/mL was also diluted with 20 mM His, pH of 5.8 to 169 mg/mL to reach 135 mM ArgHCl. The data presented in Table 3.2 indicated that the viscosity was only 10.6 centipoise (CP) even at 189 mg/mL CFI-HSA, indicating minimal risk of excessive viscosity using the tested formulation.

TABLE 3.2
Viscosity of high-concentration CFI-HSA

| CFI-HSA | Buffer | Viscosity |
|---|---|---|
| 189 mg/mL | 20 mM His, 150 mM Arg.HCl, pH of 5.8 | 10.6 CP |
| 169 mg/mL | 20 mM His, 135 mM Arg.HCl, pH of 5.8 | 8.1 CP |

The concentrated samples were 0.2 micron filtered and tested for aggregates using SEC. No change in aggregate level (1.5%) was observed in the 188.7 mg/mL sample in 150 mM arginine diluted to 5 mg/mL and stored in the 5° C. autosampler. The 169.7 mg/ml sample that was diluted to 5 mg/ml and stored in the 5° C. autosampler for 1 week showed only 1.72% aggregates. The 169.7 mg/mL sample after 1 week at 5° C. then diluted to 5 mg/ml showed 2.5% HMW species.

Bioactivity of the CFI-HSA sample concentrated to 169.7 mg/mL was assessed using an S2288 chromogenic assay. As shown in FIG. 20, the activity of the 169.7 mg/mL sample is comparable to that of the control (Ctl) sample. The minor difference in the slope of the curves could be due to either inaccuracy of the final concentration in the assay after significant dilutions of a viscous solution, or due to some loss in bioactivity after about 1 month storage at 5° C.

Taken together, these results suggest a liquid formulation containing arginine hydrochloride and histidine buffer can stabilize CFI-HSA at high concentration (e.g. greater than 150 mg/mL).

### Formulation Screening

A panel of formulations (F1 to F7) were screened to determine the effect of ionic strength, buffer type, cryoprotectant and calcium chloride (CaCl₂) on the stability of CFI-HSA. Formulations included pharmaceutically acceptable excipients, including a range of concentrations of tonicity modifiers (also referred to interchangeably herein as tonicifiers), cryoprotectants, lyoprotectants, stabilizer and surfactant were added to the previously tested histidine and arginine buffer. Table 3.3 shows the formulations tested in this example, F1-F7, not to be confused with the F1-F12 of Example 2.

TABLE 3.3
Tested formulations for CFI-HSA

| | | | Tonicifier | | | | |
|---|---|---|---|---|---|---|---|
| | | Histidine | Arginine | | | | |
| | | Buffer | Hydrochloride | Cryoprotecant/ | | Surfactant | |
| Formulation | pH | (mM) | (mM) | Lyoprotectant | Stabilizer | (PS20) | Osmolarity |
| F1 | 5.8 | 20 | 135 | — | — | 0.02% | 279 |
| F2 | 5.8 | 20 | 70 | 4% glucose | — | 0.02% | 279 |
| F3 | 5.8 | 20 | — | 8.5% sucrose | — | 0.02% | 285 |
| F4 | 5.8 | 20 | 70 | 120 mM glycine | — | 0.02% | 282 |
| F5 | 5.8 | 20 | 70 | 2.5% sorbitol | — | 0.02% | 299 |
| F6 | 5.8 | 20 | 70 | 4% trehalose | — | 0.02% | 279 |
| F7 | 5.8 | 20 | — | 5% sucrose | 35 mM | 0.02% | 279 |
| | | | | | CaCl₂ | | |

The stock CFI-HSA sample was concentrated to about 40 mg/mL, then dialyzed against the 7 formulations listed in Table 3.3. The concentrated samples were 0.2 micron filtered and aseptically filled in glass vials and capped. One vial of each composition was subjected to 3 cycles of freezing and thawing, and assessed by appearance, OD280 and SEC. For a short term thermal stability study, four vials of each composition were included in a 5 week stability study, one each at −70° C., 5° C., 25° C. and 40° C. 5-week stability samples were analyzed by OD280, SEC and CE-SDS. In addition, to assess deamidation and oxidation, peptide map MS/MS and bioactivity analysis were conducted for selected samples.

After 5 weeks, visual inspection showed that all the formulations were colloidally stable, with the exception of F3 when stored at 40° C. sample which had signs of the precipitation. Protein concentration measurements at OD280 showed the CFI-HSA concentrations remained unchanged after either three freeze/thaw cycles 1-week, or 5-week storage at all tested temperatures.

Assessment of purity by SEC and CD-SDS showed that for all 7 formulations, −70° C. samples were unchanged in purity, 5° C. samples have about 97% purity, 25° C. samples have about 96%, and all 40° C. samples are between 90-92% purity of the main species. The significant decrease in purity at 40° C. and slight decrease in purity at 25° C. over 5 weeks of storage in all 7 formulations indicate that a liquid high concentration formulation at room temperature is not feasible.

Bioactivity assays were performed to verify that CFI-HSA maintained activity following storage in the tested formulations and storage conditions (Table 3.4). The formulations are overall relatively comparable. Formulation FI is slightly more stable than the other six formulations at 40° C. Formulation F3 is the least stable at both 25° C. and 40° C., which showed 20-30% lower activity compared to −70° C. controls, and a greater loss of activity than F2, F4, or F1 samples.

TABLE 3.4
Bioactivity of CFI-HSA following tested storage conditions

| | F1 | F2 | F3 | F4 | F5 | F6 | F7 |
|---|---|---|---|---|---|---|---|
| 5° C. | 113% | 94% | 92% | 99% | 107% | 112% | 107% |
| 25° C. | 116% | 83% | 72% | 89% | 85% | 90% | — |
| 40° C. | 62% | 35% | 26% | 32% | 35% | 38% | — |

Peptide mapping MS/MS was performed for the CFI-HSA formulations to determine the level of deamidation and oxidation following the tested storage conditions. Formulations F1, F2, F4, F5, and F6 following storage for 5 weeks at 5° C. samples were tested. An F4 (F/T) frozen was run as a control. The results indicated that deamidation and oxidation levels on all sites are comparable among different formulations.

Additional testing and formulations developed for clinical evaluation and drug manufacturing include formulations in Table 3.5. CFI-HSA was buffer exchanged to the 3 new formulations in Table 3.5 and concentrated to 100 mg/mL or 150 mg/mL while observing any precipitation, severe opalescence, or high viscosity. To make the formulations more suitable for lyophilized product, 60 mM glycine or 60 mM mannitol were added to F2 and F3 to protect the structure of the lyophilized cake, respectively. Samples were then sterile filtered and file, and tested for viscosity, SEC and activity.

TABLE 3.5
Formulations for clinical manufacturing and testing

| | Prot. Conc. | | | | | | |
|---|---|---|---|---|---|---|---|
| | (mg/mL) | pH | Buffer | Tonicifier/Cyro/Lyoprotectant | | | PS-80 |
| F1 | 100 | 5.8 | 20 mM | 135 mM Arg HCL | | | 0.02% |
| F2 | | | Histidine | 70 mM Arg HCL | 2% sucrose | 60 mM Glycine | 0.02% |
| F3 | | | | 70 mM Arg HCL | 2% sucrose | 60 mM Mannitol | 0.02% |
| F1 | 150 | | | 135 mM Arg HCL | | | 0.02% |
| F2 | | | | 70 mM Arg HCL | 2% sucrose | 60 mM Glycine | 0.02% |
| F3 | | | | 70 mM Arg HCL | 2% sucrose | 60 mM Mannitol | 0.02% |

1. A formulation comprising a wild type complement factor I (CFI) or a variant thereof (CFI variant), wherein the formulation comprises a buffering agent, a surfactant, and a tonicity modifier.

2. The formulation of claim 1, wherein the formulation comprises one or more of a bulking agent, a cryoprotectant, and a lyoprotectant.

3. The formulation of claim 1, wherein the concentration of the CFI or variant thereof is present at about 10 mg/ml to about 300 mg/ml.

4. (canceled)

5. The formulation of claim 1, wherein the buffering agent comprises acetate, histidine, or succinate.

6. (canceled)

7. The formulation of claim 1, wherein the buffering agent is present at about 1 mM to about 50 mM.

8. The formulation of claim 1, wherein the surfactant is polysorbate 80, polysorbate 20, polysorbate 80/20, or poloxamer F68.

9. The formulation of claim 8, wherein the surfactant is present at about 0.001% to about 0.1% v/v.

10-11. (canceled)

12. The formulation of claim 1, wherein the tonicity modifier is sorbitol, trehalose, sodium chloride, or arginine hydrochloride.

13. The formulation of claim 1, wherein the bulking agent is trehalose or glycine.

14. The formulation of claim 1, wherein the tonicity modifier is selected from:
(i) sorbitol, wherein the sorbitol is present at about 1% to about 10% v/v;
(ii) trehalose, wherein the trehalose is present at about 1% to about 15% v/v;
(iii) sodium chloride, wherein the sodium chloride is present at about 1 mM to about 500 mM; and
(iv) arginine hydrochloride, wherein the arginine hydrochloride is present at about 10 mM to about 200 mM.

15-22. (canceled)

23. The formulation of claim 13, wherein the glycine is present at about 1% to about 5% v/v or about 1 mM to about 100 mM.

24-26. (canceled)

27. The formulation of claim 2, wherein the cryoprotectant or lyoprotectant is selected from glucose, sucrose, glycine, sorbitol, trehalose, sucrose, or mannitol.

28. (canceled)

29. The formulation of claim 28, wherein;
(a) the glucose is present at about 4% v/v;
(b) the sucrose is present at about 1% to about 10% v/v;
(c) the glycine is present at about 50 mM to about 150 mM;
(d) the trehalose is present at about 1% to about 10% v/v;
(e) the mannitol is present at about 1 mM to about 100 mM; or
(f) the sorbitol is present at about 1% to about 5% v/v.

30-41. (canceled)

42. The formulation of claim 1, wherein the formulation is selected from the formulations presented in Table 2.2 or Table 3.3.

43. The formulation of claim 1, wherein the formulation is in a solid form, a lyophilized form, or a liquid form.

44-45. (canceled)

46. The formulation of claim 1, wherein the formulation is stable at any one or more of −80° C.+/−2° C., −20° C.+/−2° C., 0° C.+/−2° C., 4° C.+/−2° C., 25° C.+/−2° C., 37° C.+/−2° C., 45° C.+/−2° C., or at 60° C.+/−2° C.

47. The formulation of claim 1, wherein the formulation is stable for at least 1 week, for at least one month, or at least one year.

48. The formulation of claim 1, wherein the formulation comprises wild type CFI.

49. The formulation of claim 1, wherein the formulation comprises a CFI variant comprising at least one modification with respect to a wild type CFI, wherein the CFI variant is capable of modulating the complement system, and wherein the CFI variant has at least one improved characteristic as compared to the wild type CFI.

50-53. (canceled)

54. The formulation of claim 49, wherein the CFI variant is selected from those presented in Table 2.

55. The formulation of claim 1, wherein the wild type CFI or CFI variant is a first component of a fusion construct comprising a first component and at least a second component, and the wild type CFI or CFI variant is fused to the second component.

56-61. (canceled)

62. A formulation comprising a fusion construct comprising a wild type CFI or variant thereof (CFI variant), where the wild type CFI or CFI variant is fused to a human serum albumin, and the formulation is selected from those presented in Table 2.2 or Table 3.3.

63. The formulation of claim 62, wherein the fusion construct comprises the amino acid sequence of SEQ ID NO: 21.

64. A method of treating a condition in a subject in need thereof, the method comprising administering to the subject a therapeutically effective amount of the formulation of claim 1.

65-69. (canceled)
